(19) United States
(12) Patent Application Publication
(10) Pub. No.: US 2025/0325303 A1
(43) Pub. Date: Oct. 23, 2025

(54) Cervical Navigator System and Method of Use Thereof

(71) Applicant: Gynovent LLC, Cheyenne, WY (US)

(72) Inventors: Jennifer Claman, EL SEGUNDO, CA (US); Noah Bruckner, Marietta, GA (US); Alana Frankel, EL SEGUNDO, CA (US); Isabelle De Bruyker, EL SEGUNDO, CA (US); Sarah Fraga, EL SEGUNDO, CA (US); Brody Smith, EL SEGUNDO, CA (US)

(21) Appl. No.: 19/186,498

(22) Filed: Apr. 22, 2025

Related U.S. Application Data
(60) Provisional application No. 63/637,175, filed on Apr. 22, 2024.
(60) Provisional application No. 63/638,882, filed on Apr. 25, 2024.

(51) Int. Cl. A61B 17/42; A61B 1/32; A61B 17/00; A61B 90/35; A61M 29/02

(52) U.S. Cl. CPC A61B 17/4241; A61B 1/32; A61B 90/35; A61M 29/02; A61B 2017/00557; A61B 2017/4225; A61M 2210/1433

(57) ABSTRACT

A cervical navigator, including an elongate body having a rigid structure that at least partially deforms in response to contact with living tissue, and an opening disposed through a center portion of the elongate body and at least one dilator removably disposed through the opening that increases the size of an elongate body in response to insertion of at least one dilator into the opening and at least partial movement through the opening.

### CROSS-REFERENCE TO RELATED APPLICATION

This application incorporates by reference and claims the benefit of priority to U.S. Provisional Application 63/637,175 filed on Apr. 22, 2024, and U.S. Provisional Application 63/638,882 filed on Apr. 25, 2024, incorporated hereby in its entirety.

### BACKGROUND OF THE INVENTION

### 1. Field of the Invention

The present disclosure relates to a medical system. More specifically, the present disclosure relates to a gynecological cervical navigator system having an anchoring portion to anchor the medical system in a passage of a human body while a central portion of the medical system performs dilation or additional therapeutic treatment in the passage, and a vaginal retraction component that provides visualization and access to the cervix.

### 2. Description of the Related Art

Cervical dilation is the process of expanding an individual's cervix, allowing entry into the uterus. A variety of procedures may require cervical dilation, including oophorectomies, myomectomies, miscarriage evacuations, cervical cancer screenings, heavy menstrual bleeding, endometrial ablations, abortions, hysteroscopies, presence of post-menopausal bleeding, uterine cancer not diagnosed sufficiently with endometrial biopsy, and thickened uterine lining. Hysteroscopy is the most frequently performed procedure that requires cervical dilation. To illustrate the potential dangers of cervical dilation, about 50% of hysteroscopic complications (perforations, false passageway, and tears) are directly related to difficulty with cervical entry as the cervix must be dilated to the size of the hysteroscope to allow the physician to visualize the uterus.

Current cervical dilation processes involve plastic or metal dilators of differing diameters to enter the cervix until the desired dilation size is achieved. However, using stiff dilators can cause perforations of the cervix, uterus, and other surrounding organs and can separately lead to the creation of false cavities in the cervical canal. Each time an increasing size dilator is passed through the cervix, the provider has to re-navigate the cervical passageway. Thereby, multiplying the risks of cervical navigation, and increasing the risk for patient injury, by each new entry attempt. New methods of cervical dilation are needed as uterine perforations occur in 0.1% to 4% of all procedures. While these percentages are small, many perforations are not identified or underreported. Additionally, perforations can lead to life-threatening consequences, including small bowel obstruction, uterine rupture, intrauterine adhesions, and infertility. And almost universally, when identified, precludes completion of the scheduled evaluation or procedure.

False passageway creation occurs when the dilator enters an unintended space and is more common in patients with tortuous cervixes or when they suffer from cervical stenosis. False passageway creation can also occur if a patient has abnormal uterine positions, including if the uterus is in an acutely anteflexed, anteverted, or retroverted position.

What is needed, therefore, is an improved device and method for cervix dilation that substantially reduces the discomfort and the risk of perforations to the patients.

Before explaining the various embodiments of the invention in detail, it is to be understood that the invention is not limited in its application to the details of construction and to the arrangements of the components set forth in the following description or illustrated in the drawings. Rather, the invention is capable of other embodiments and of being practiced and carried out in various ways. Also, it is to be understood that the terminology employed herein is for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

For a better understanding of the invention, its operating advantages and the specific objects attained by its uses, reference should be made to the accompanying drawings and descriptive matter in which there are illustrated preferred embodiments of the invention.

Various objects, features, aspects and advantages of the present embodiment will become more apparent from the following detailed description of embodiments of the embodiment, along with the accompanying drawings in which like numerals represent like components.

### SUMMARY OF THE INVENTION

In one aspect, the present invention is embodied as a gynecological cervical navigator system comprising an anchoring portion to anchor the medical system in a passage of a human body while a central portion of the medical system performs dilation or additional therapeutic treatment in the passage, and a vaginal retraction component that provides visualization and access to the cervix.

In one embodiment, the present disclosure is embodied as a medical device comprising a cervical dilator component with a plurality of sequential dilators that cascade over one another, allowing for progressive cervical dilation without the need to reestablish a path through the cervix with each increasing dilator size.

In another embodiment, the disclosure is embodied as a posterior vaginal wall retractor that maintains access and visualization of the cervix without repetitive insertion and removal of a speculum, wherein the retractor component may be used separately or in conjunction with the cervical dilator component.

In an alternative embodiment, the inflation unit is a hollow, flat, donut-shaped balloon that anchors the device at the internal cervical ostia while allowing passage of instruments through its center opening, and when traction is applied to the device, maintains access to the cervix and prevents efflux of uterine distending medium.

In accordance with one form of this disclosure, there is provided a method for cervical dilation comprising placing an initial small dilator through the cervix to establish a pathway, inflating a balloon to secure the device at the level of the internal cervical ostia, and sequentially advancing dilators of increasing diameter through the secured pathway without needing to reestablish the cervical path for each dilator. The sequential cervical dilators may also be used to fully dilate the cervix to the desired dilation before placement or activation of the balloon device.

Before explaining the various embodiments of the invention in detail, it is to be understood that the invention is not limited in its application to the details of construction and to the arrangements of the components set forth in the following description or illustrated in the drawings. Rather, the invention is capable of other embodiments and of being practiced and carried out in various ways. Also, it is to be understood that the terminology employed herein is for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

For a better understanding of the invention, its operating advantages and the specific objects attained by its uses, reference should be made to the accompanying drawings and descriptive matter in which there are illustrated preferred embodiments of the invention.

Various objects, features, aspects and advantages of the present embodiment will become more apparent from the following detailed description of embodiments of the embodiment, along with the accompanying drawings in which like numerals represent like components.

### BRIEF DESCRIPTION OF THE DRAWINGS

The present disclosure may be better understood, and its numerous features and advantages made apparent and readily appreciated to those skilled in the art, by referencing the accompanying drawings.

FIG. 1 is a cross-sectional view of a medical system according to one embodiment of the present disclosure showing the first embodiment of the cervical navigator with an expandable outer sheath and initial inner dilator.

FIG. 2 is a cross-sectional view of the medical system showing one embodiment after placement, with the inflation unit inflated.

FIG. 3 is a cross-sectional view of the medical system showing another embodiment with flexible dilators within the expandable outer sheath.

FIG. 4 is a cross-sectional view of the medical system showing one embodiment of the cervical navigator with a dilating device featuring a curved handle and sequential telescoping dilators that cascade over each other.

FIG. 5 is a cross-sectional view of the medical system showing another embodiment with the inflatable balloon anchoring system.

FIG. 6 is a cross-sectional view of the medical system showing the second embodiment after removal of the dilating mechanism, with the expandable outer sheath remaining in place anchored by the inflation unit, and the retractor component maintaining vaginal access.

FIG. 7A is an elevational side view of a medical system including a cervical navigator, a retractor, and obturator in accordance with some embodiments.

FIG. 7B is an elevational front view of the medical system in accordance with some embodiments.

FIG. 7C is an elevational rear view of the medical system in accordance with some embodiments.

FIG. 8 is a cross-sectional view of the medical system in accordance with some embodiments.

FIG. 9A is a cross-sectional view of the medical system including an inflation unit as disposed in the retractor in accordance with some embodiments.

FIG. 9B is a cross-sectional side view of the medical system after inflating the inflation unit in accordance with some embodiments.

FIG. 9C is a zoomed-in cross-sectional side view of the inflation unit after inflation in accordance with some embodiments.

FIG. 9D is a cross-sectional side view of the medical system after inflating the inflation unit as disposed in a cervix in accordance with some embodiments.

FIG. 10A is an elevational top view of the medical system in accordance with some embodiments.

FIG. 10B is a perspective top view of the medical system in accordance with some embodiments.

FIG. 10C is a perspective top view of the medical system after inflating the inflation unit in accordance with some embodiments.

FIG. 11A is a cross-sectional side view of a cervical navigator in accordance with some embodiments.

FIG. 11B is a cross-sectional top view of the cervical navigator in accordance with some embodiments

FIG. 11C is a perspective top view of the cervical navigator in accordance with some embodiments.

FIG. 12A is an elevational rear view of a posterior vaginal wall retractor in accordance with some embodiments.

FIG. 12B is an elevational front view of the posterior vaginal wall retractor in accordance with some embodiments.

FIG. 12C is a cross-sectional side view of the posterior vaginal wall retractor in accordance with some embodiments.

FIG. 12D is an elevational top view of the posterior vaginal wall retractor in accordance with some embodiments.

FIG. 13A is a side view of a dilator in accordance with some embodiments.

FIG. 13B is a top view of a dilator in accordance with some embodiments.

FIG. 13C is a rear view of a dilator in accordance with some embodiments.

FIG. 14 is a perspective side view of a cervical navigator device in accordance with some embodiments.

FIG. 15 is a perspective front view of the cervical navigator device in accordance with some embodiments.

FIG. 16 is a zoomed-in view of a lever unit of the cervical navigator device in accordance with some embodiments.

FIG. 17 is a zoomed-in view of a plurality of dilators in accordance with some embodiments.

FIG. 18A is a cross-sectional side view of the plurality of dilators in accordance with some embodiments.

FIG. 18B is a cross-sectional side view of the plurality of dilators in accordance with some embodiments.

FIG. 18C is a cross-sectional side view of the plurality of dilators in accordance with some embodiments.

FIG. 18D is a cross-sectional side view of the plurality of dilators in accordance with some embodiments.

FIG. 18E is a cross-sectional side view of the plurality of dilators in accordance with some embodiments.

FIG. 19 is a cross-sectional side view of the lever unit of the cervical navigator device in accordance with some embodiments.

FIG. 20 is a perspective top view of the assembled medical system in accordance with some embodiments.

FIG. 21 is cross-sectional view of the assembled medical system in accordance with some embodiments.

FIG. 22 is a rear overlay view of the assembled medical system in accordance with some embodiments.

FIG. 23 is a front overlay view of the assembled medical system in accordance with some embodiments.

The same elements or parts throughout the figures of the drawings are designated by the same reference characters.

### DETAILED DESCRIPTION OF THE INVENTION

The embodiment and various embodiments can now be better understood by turning to the following detailed description of the embodiments, which are presented as illustrated examples of the embodiment defined in the claims. It is expressly understood that the embodiment as defined by the claims may be broader than the illustrated embodiments described below. Many alterations and modifications may be made by those having ordinary skill in the art without departing from the spirit and scope of the embodiments.

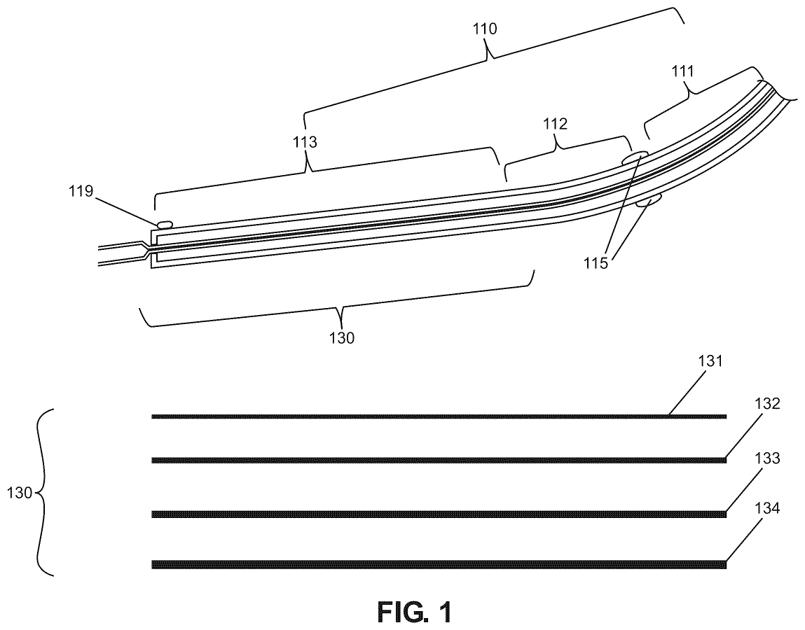

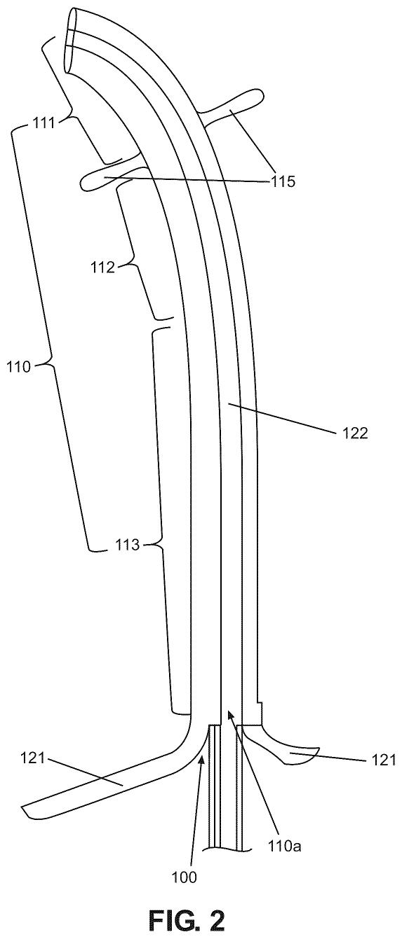

FIG. 1 is a cross-sectional view of a medical system showing the first embodiment of the cervical navigator during insertion, with the outer sheath containing the initial dilator and the inflation unit in a deflated state, positioned for passage through the vagina and cervix into the uterus. FIG. 2 is a cross-sectional view of the medical system showing the first embodiment after proper placement, with the inflation unit inflated at the internal cervical ostia to anchor the device, and the retractor positioned in its handle orientation to provide direct visualization and access to the cervix.

In some embodiments, a medical system 100 includes a cervical navigator 110. The medical system 100 is to be inserted at least partly in the uterus through the vagina and cervix, wherein cervical dilation will be performed to expand the patient's cervix and allow easier entry into the uterus. In some embodiments, the cervical navigator 110 is removably inserted into a body cavity. For example, the cervical navigator 110 is inserted into a portion of a vagina, a portion of a cervix, and a portion of a uterus. After the cervical navigator 110 is inserted into the uterus, the cervical navigator 110 is dilated. Sequential dilators are passed within the cervix expanding the cervix and facilitating access to the uterine cavity. As such, the cervical navigator 110 facilitates medical examination and/or operation within the body cavity of a patient.

In some embodiments, as shown in FIGS. 1, 2 and 3, the device consists of an outer sheath which is connected to a handle component which sits outside the patient. In this embodiment, the outer sheath is an expandable material, soft plastic, rubber or silicone. The device, in its smallest diameter with the smallest initial dilator in place, is initially inserted and guided along the vagina and through the cervix. The cervical dilator 110 contains the expandable outer sheath and the initial inner dilator. Once successfully inserted through the cervix, the balloon 115 is inflated and the device is pulled back to the internal cervical ostia. The balloon is shaped like a donut in that it has an open center to facilitate the passage of instruments into the uterus. It is flat enough and wide enough to not obscure access or visualization to the uterus and to sufficiently occlude the cervix and prevent efflux of uterine distending medium and to secure the device to the cervix when traction is applied to the handle. Sequential dilators of increasing size are then passed through the secured, expandable cervical dilator 110 until the desired cervical dilation is achieved. In this way, the passageway into the uterus is identified and secured with a very small dilator and that “tract” is maintained by the outer sheath which is secured in place so that sequential dilators follow the same course without having to re-navigate the actual passageway.

The cervical dilator 110 includes a first portion 111, a second portion 112, a third portion 113, and an inflation unit 115, but is not limited thereto. The first portion 111, the second portion 112, and the third portion 113 are configured to be placed in different portions of the patient's body cavity. The first portion 111 is configured to be placed in the patient's uterus. The second portion 112 is configured to be placed in the patient's cervix or cervical passage. The third portion 113 is configured to be placed in the patient's vagina. Collectively, the first portion 111, a second portion 112, and a third portion 113 is also referred to as an elongated body 111, 112, 113. That is, the elongate body 111, 112, 113 has an elongate shape and has a rigid structure in absence of an application of force (e.g., pushing, pulling). However, the elongated body 111, 112, 113 at least partially deforms (e.g., bends, stretches, expands) in response to contact with living tissue (e.g., the vagina, the cervix, the uterus). In different embodiments, the cervical dilator 110 further includes a fourth portion connected to the third portion 113 and extension outside the patient's body cavity.

In the depicted example of FIG. 1, the medical system 100 is located in the vagina, cervix, and uterus of the patient except for the very small portion of a posterior vaginal wall retractor 120 (hereinafter, the retractor 120) outside the vagina and just located at the introitus. The retractor 120 is removably connected to the elongate body 111, 112, 113. Specifically, in some embodiments, the retractor 120 includes a plurality of protrusions (not shown in FIGS. 1 and 2) that connect to a plurality of grooves disposed on the sequential dilating device 141 that reversibly connects and secures the two components together. In other words, the plurality of protrusions corresponds to the plurality of grooves and connect the retractor 120 to the cervical dilator 110. The retractor 120 is disposed at an end of the cervical dilator 110. The retractor 120 comes out the vagina and sits on the perineum going towards the rectum. The retractor 120 is used to open up the vaginal passageway to visualize the obturator within the cervical dilator 110 for passage of a hysteroscope or other medical instruments into the uterus. Additionally, the retractor 120 facilitates gripping and manipulation of the outer sheath 110. When the retractor is secured with the balloon, it also allows for traction on the cervix without the placement of additional devices, thereby straightening the uterus and decreasing perforation risk.

In the depicted example of FIGS. 1 and 2, the plurality of dilators 130 includes a first dilator 131 with a 3 mm diameter, a second dilator 132 with a 5 mm diameter, a third dilator 133 with a 7 mm diameter, and a fourth dilator 134 with a 9 mm diameter. Each of the plurality of dilators 130 are flexible and preferably made of polymer such as silicon, plastics such as polypropylene, or other flexible materials. The plurality of dilators 130 are constructed to have enough rigidity to navigate in the cervical dilator 110 without breaking. Further, each of the plurality of dilators 130 is preferably made of materials with relatively high texture smoothness to prevent damage (e.g., perforating) to the cervical dilator 110 during insertion and/or movement within the cervical dilator 110.

After the elongated body 111, 112, 113 of the cervical dilators 110 is inserted in the uterus, cervix, and vagina, the inflation unit 115 is inflated to anchor the cervical dilator 110 within the uterus. Accordingly, the inflation unit 115 is perpendicularly disposed within the uterus with respect to the elongate body 111, 112, 113 to prevent extraction of the elongate body 111, 112, 113 from the uterus, the cervix, and the vagina. Subsequently, at least one of the plurality of dilators 130 is inserted in the cervical dilator 110 through the first opening 110a to pass through the second portion 112 and dilate the cervix. The cervix dilation procedure involves gradually and progressively dilating the cervix to avoid damage to the patient's cervix. The cervix dilation procedure starts by inserting a cervical dilator with the smallest diameter, i.e. the first dilator 131, in the cervical dilator 110 to pass through the second portion 112 and dilate the cervix in the process. The first dilator 131 is then removed from the cervical dilator 110 and replaced with the second dilator 132 to pass through the second portion 112 and again dilate the cervix in the process. Please note that different procedures require different degrees of cervical dilation. Some will require 3 mm, some 5 mm, some 7 mm and some 9 mm; the device facilitates any of these dilations. Thus, not all cervical dilation procedures require the use of all of the plurality of dilators 131-134. Further, in the present embodiments, the medical system 100 includes four cervical dilators 131-134 with diameters ranging from 3 mm to 9 mm. In different embodiments, the medical system 100 can include other numbers of dilators with diameters other than 3 mm, 5 mm, 7 mm, and 9 mm. For example, in different embodiments, the medical system 1 includes dilators with smaller diameters or larger diameters than the diameters of the plurality of dilators 131-134. After the cervix dilation procedure is complete, the plurality of dilators 131-134 or whatever number of dilators were used are extracted (i.e., removed, withdrawn) from the cervical dilator 110. After extraction, the cervical dilator 110 maintains the increased size (i.e., expanded shape, increased diameter, increased circumference). Alternatively, the cervical dilator 110 decreases in size (i.e., retracts) back to its original shape after the dilators are removed and retains capacity to increase in size in response to insertion of additional medical instruments, such as a hysteroscope.

The first dilator 131 has a distal portion with a diameter of approximately 1 mm and configured to be inserted in the cervical dilator 110 through the first opening 110a (illustrated in FIG. 2) and then push the first portion 111 toward the uterus while passing through the vagina and cervix. The retractor 120 is movably connected to the cervical dilator 110 and configured to switch between a flat orientation (illustrated in FIG. 1) and a handle orientation (illustrated in FIG. 2) before and after the outer sheath 110 is properly placed and anchored in the patient's body cavity. Before the cervical dilator 110 is inserted and properly anchored in the patient's uterus, the retractor 120 is preferably placed in the flat orientation as illustrated in FIG. 1 which makes it easier for the physician to hold the retractor 120. Once the cervical dilator 110 is properly anchored in the uterus, the handle can be bent and split into two pieces and create a second opening (illustrated in FIG. 2) connecting the first opening 110a for the physician to see the cervix without having to use a speculum, wherein at least one part of the bent retractor 120 covers the perineum of the patient.

The inflation unit 115 is located between the first and second portions 111, 112 and is configured to anchor the cervical dilator 110 and maintain the elongate body 111, 112, 113 in the corresponding positions in the patient's body cavity. As depicted in FIG. 1, the inflation unit 115 is deflated and laid down on an outer surface of the cervical dilator 110, so that while pushing the cervical dilator 110 through the cervix, the inflation unit 115 will not collide with the cervix and hinder the movement of the first portion 111 through the cervix to reach the uterus.

In some embodiments, the inflation unit 115 is inflatable and can be inflated to become perpendicular to the cervical dilator 110 as illustrated in FIG. 2. The combined width of the cervical dilator 110 and the inflation unit 115 is greater than the width of the passage of the cervix. Thus, the inflation unit 115 prevents the first portion 111 from being retracted back into the cervix by an external force (e.g., pulling). Further, as illustrated in FIG. 1, the inflation unit 115 has an inflation port 119 disposed on the cervical dilator 110 and connected to the inflation unit 115 to receive liquid or air in for inflating the inflation unit 115. The inflation port 119 is configured to be selectively opened for receiving liquid or air and sealed once the inflation of inflation unit 115 is complete. While initially, more of the medical system 100 is within the vagina and passes into the uterus, after the inflation unit 115 is inflated, the retractor 120 may be withdrawn somewhat to facilitate retraction of the posterior vagina and perineum.

The first goal of the cervix dilation procedure is to insert the first portion 111 in the uterus and place the inflation unit 115 at the isthmus, also known as the internal cervical ostia (a.k.a., internal os), which is the opening that leads to the uterus. The internal os is the upper boundary of the cervix, which is the lower third of the uterus. The internal os connects the body of the uterus to the passage of the cervix, which is called the endocervical canal. When the inflation unit 115 reaches the internal os, the inflation unit 115 is inflated to be substantially perpendicular to the outer surface of the cervical dilator 110 as illustrated in FIG. 2. At this moment, the combined width of the cervical dilator 110 and the inflation unit 115 while inflated is greater than the width of the cervix passage, such that the inflation unit 115 serves as an obstacle to prevent the first portion 111 from being retracted back in the cervix. In other words, the inflation unit 115 is a dilatable balloon anchoring system at the level of the internal cervical os. Once the cervical dilator 110 is secured, it can be used to facilitate the passage of instruments, such as a hysteroscope, into the uterus.

The inflation unit 115 preferably has a minimal thickness and width so that it will not collide with the cervix and unnecessarily hinder the movement of the first portion 111 through the cervix to reach the uterus. Also, the inflation unit 115 is configured to have a flatten disc shape with less curve in order to better serve as obstacles to prevent the first portion 111 from being retracted back in the cervix. In different embodiments, the inflation unit 115 is configured to have an oval, sphere, or other shapes.

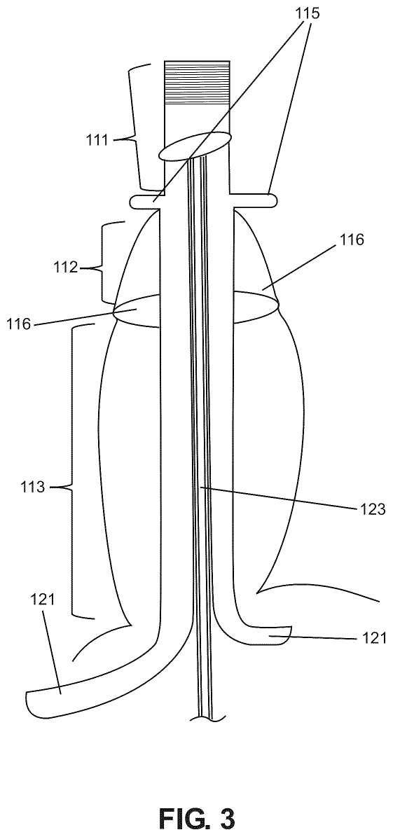

FIG. 3 is a cross-sectional view of the medical system 1 in accordance with some embodiments. In the depicted example of FIG. 3, the external cervical ostia (a.k.a., external os). The external os is an opening between the cervix and the vagina. The area labeled as 112 represents the cervix from the external to the internal cervical ostia. The inflation unit 115 is located at the internal os and anchors the cervical dilator 110, maintaining the elongate body 111, 112, 113 in the corresponding positions (i.e., uterus, cervix, vagina) in the patient's body cavity.

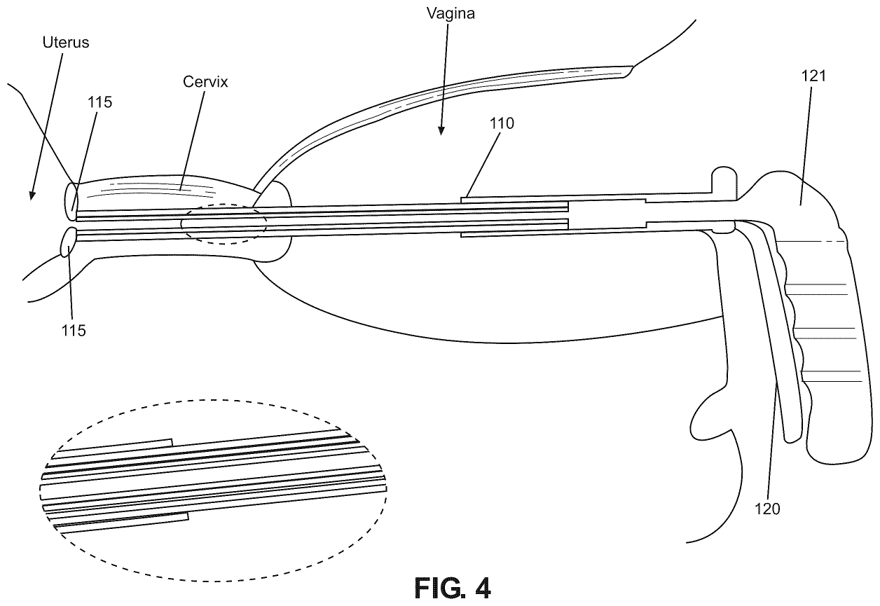

FIG. 4 is a cross-sectional view of the medical system 1 in accordance with some embodiments.

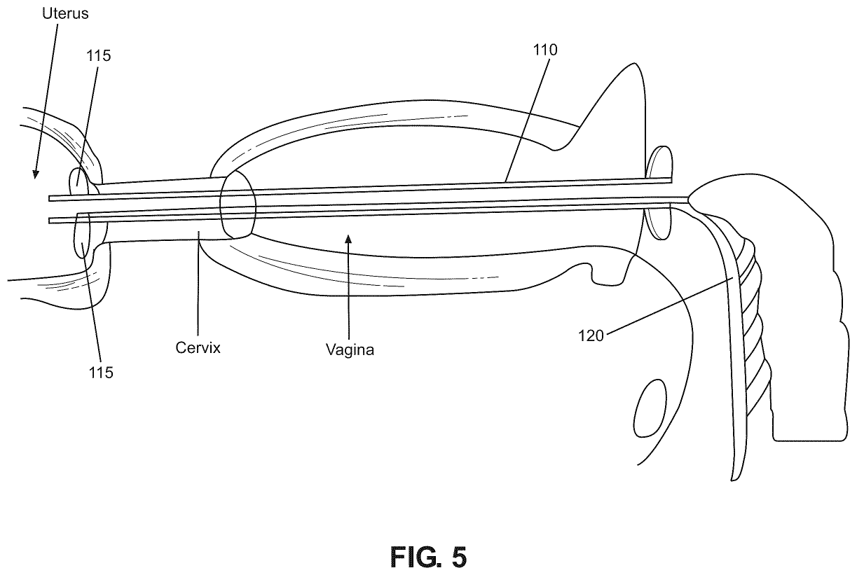

FIG. 5 is a cross-sectional view of the medical system 1 in accordance with some embodiments.

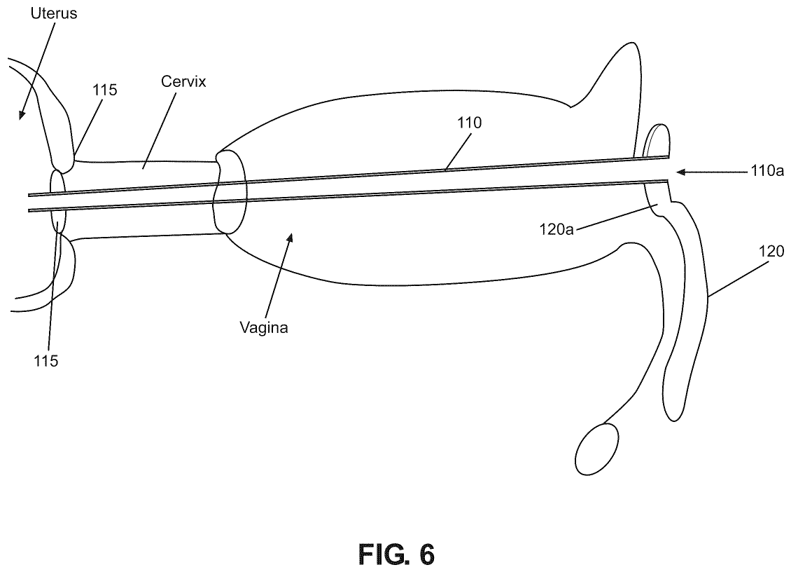

FIG. 6 is a cross-sectional view of the medical system in accordance with some embodiments. In some embodiments, the medical system 100 includes a cervical dilator 110, a retractor 120, and a plurality of dilators (a.k.a., a dilating device). The dilating device is shown in FIGS. 4 and 5, while FIG. 6 shows the cervical dilator without the dilating device after it has been removed. The cervical dilator 110 of this embodiment is substantially identical to its counterparts of the embodiments discussed above. The retractor 120 is made of harder materials including polymer such as silicon, plastics such as polypropylene, or other flexible materials. As illustrated in FIGS. 4-6, the cervical dilator 110 and the retractor 120 are connected to each other, wherein the retractor 120 covers the perineum of the patient. The cervical dilator 110 is made of soft and expandable materials such as rubber latex, synthetic, and natural membrane in this embodiment. The cervical dilator 110 is essentially a flexible balloon and has a first opening 110a configured to enable other medical devices to enter the interior of the cervical dilator 110 and expand the cervical dilator 110 outward from within. This is one embodiment of the device. In later embodiments, the vaginal retractor component is separate from the cervical dilator component. The cervical dilator in this embodiment can be made of plastic, silicone or some component of overlay with the dilators as flexible rods.

In some embodiments, the dilating device includes a dilating device handle, and a dilator portion connected to each other. The dilating device handle is a handle for operating a telescoping dilating mechanism. Referring to FIG. 4, the dilating device handle is curved with respect to a direction and sits within, and ultimately detaches from, a portion of the outer sheath 110 that is disposed outside the vagina and retracting the posterior vaginal wall. The dilator portion is configured to be inserted in the cervical dilator 110. In this embodiment, the cervix may be dilated with the cascading dilators before the outer sheath is secured. The dilator portion is then inserted into a second portion (e.g., corresponding to the second portion 112 discussed above with respect to FIGS. 1-3) of the cervical dilator 110 to dilate the cervix surrounding the second portion 112.

Referring again to FIG. 4, the dilator portion includes a plurality of dilator portions having different diameters and configured to be sequentially inserted in the second portion of the cervical dilator 110 to dilate the cervix. The dilator portion includes a first dilator portion with a 1 mm diameter, a second dilator portion with a 3 mm diameter, a third dilator portion with a 5 mm diameter, and a fourth dilator portion with a 7 mm diameter. In some embodiments, the dilator portions are telescopic tubes that are each separately movable over other dilator portions. To illustrate via an example, the first dilator portion is extended into the cervix. Thereafter, the second dilator portion is extended is response to manipulation (e.g., pushing, pulling, rotating a knob, rotating a lever, etc.) of the dilating device handle, such that the second dilator portion telescopically extends over and surrounds the first dilator portion. Accordingly, the second dilator tube has an opening and a channel at a center portion with a size (e.g., diameter, length) that corresponds to a size (e.g., diameter, length) of the first dilator portion. Specifically, the size of the opening and the channel of the second dilator tube is greater than the size of the first dilator portion to facilitate extending the second dilator tube over and surrounding the first dilator portion.

The first goal of the cervix dilation procedure in this embodiment is to insert the first portion (e.g., corresponding to the first portion 111 discussed above with respect to FIGS. 1-3) in the uterus by the first dilator portion and place the inflation unit 115 at the internal os. When the inflation unit 115 reaches the internal os it can be inflated to be positioned against the internal cervical os. At this moment, the combined width of the cervical dilator 110 and the inflation unit 115 is greater than the width of the cervix passage, wherein the inflation unit 115 now serves as an obstacle to prevent the first portion from being retracted back in the cervix. In some embodiments, the cascading dilators are advanced within the cervix to complete the desired cervical dilation before the balloon is inflated and secured.

Once an elongate body of the cervical dilator 110 is properly placed in the uterus, cervix, and vagina and the inflation unit 115 properly anchors the cervical dilator 110 at the internal os, the dilator portions are inserted in the cervical dilator 110 to pass through the second portion and dilate the cervix. The cervix dilation procedure involves gradually and progressively dilating the cervix to avoid damage to the patient's cervix. The cervix dilation procedure starts by inserting a cervical dilator with the smallest diameter, e.g., the second dilator, in the cervical dilator 110 to pass through the second portion and dilate the cervix in the process. The third dilator portion is then inserted in the second portion to dilate the cervix and then the fourth dilator portion. It is important to note that different procedures require different degrees of cervical dilation, and it may not be necessary to dilate a patient's cervix all the way to a diameter of 9 mm. Thus, not all cervical dilation procedures require the use of all dilator portions. Further, in some embodiments, the medical system 100 includes four cervical dilators with diameters ranging from 1 mm to 7 mm. In different embodiments, the medical system 1 can include other numbers of dilators with diameters other than 1 mm, 3 mm, 5 mm, and 7 mm. For example, in different embodiments, the medical system 1 includes dilators with smaller diameters or larger diameters than the diameters of the cervical dilators. After the cervix dilation procedure is complete, the cervical dilators or whatever number of dilators were used are extracted (i.e., removed, withdrawn) from the cervical navigator 110. After extraction, the cervical navigator 110 may return to its original size, but retains the capacity to expand for the introduction of additional instruments such as the hysteroscope. Here the increased size refers to at least one for an expanded shape, increased diameter, and/or an increased circumference.

In some embodiments, the cascading dilators are inserted into the cervix, the dilating mechanism is removed and the balloon anchoring system is inserted via the retractor portion which is left in place.

In the depicted example of FIGS. 5 and 6, the dilating device is removed from the cervical dilator 110 that still remains inside the patient's body cavity and then is replaced with a different dilating device having telescoping dilator portions with different diameters. The cervical dilator 110 still has the retractor 120 which is retracting the posterior vagina and perineum and the operating channel 110a for passage of instruments into the uterus. The cervical dilator 110 is traversing the vagina and passing through the cervix. The cervical dilator 110 and the retractor 120 are connected at the junction.

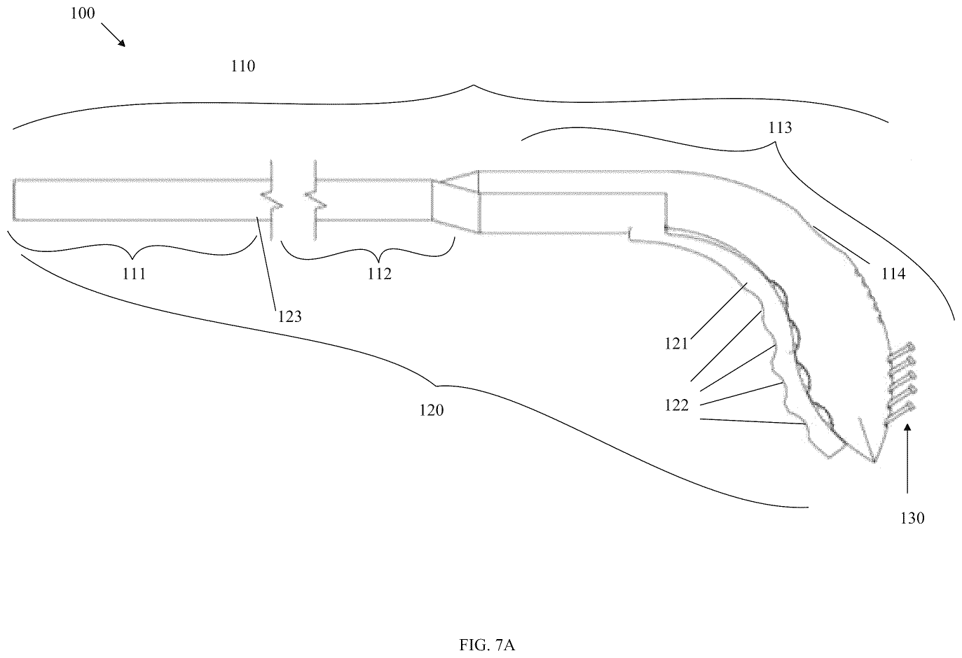

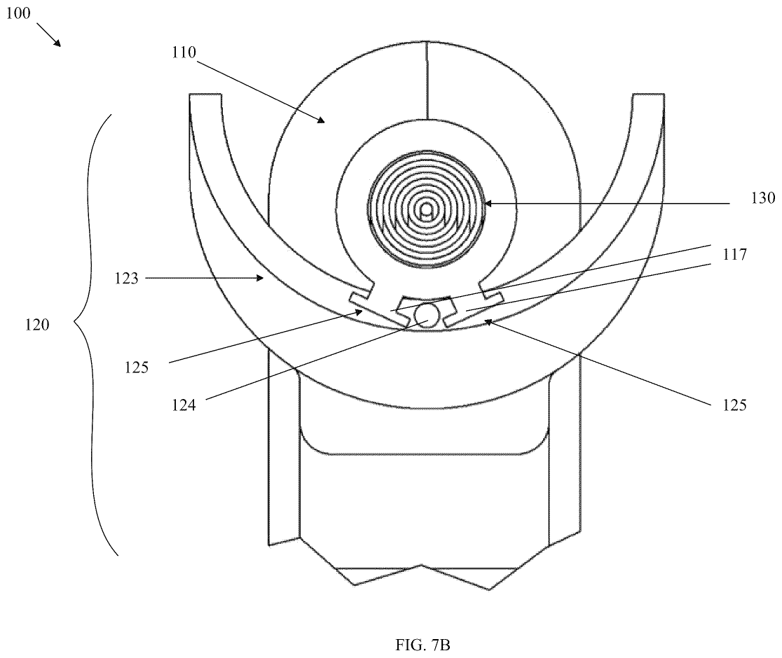

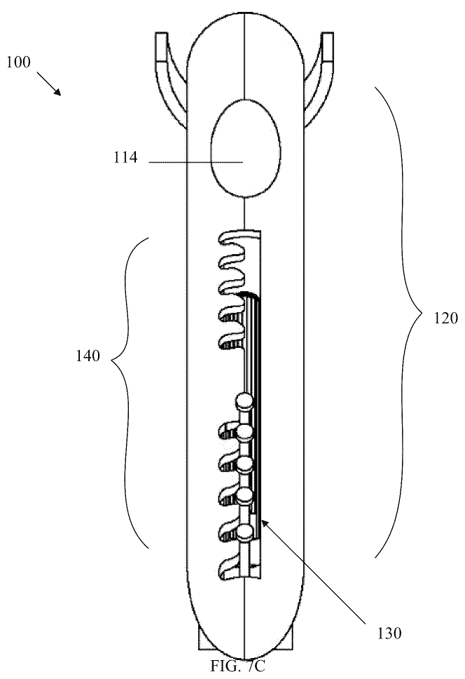

FIG. 7A is an elevational side view of a medical system 100 including a cervical navigator 110, a retractor 120, and an obturator in accordance with some embodiments.

FIG. 7B is an elevational front view of the medical system 100 in accordance with some embodiments.

FIG. 7C is an elevational rear view of the medical system 100 in accordance with some embodiments.

In the depicted example of FIGS. 7A-7C, in some embodiments, the medical system 100 includes a cervical navigator 110, a posterior vaginal wall retractor 120 (hereinafter, retractor 120), and a plurality of dilators 130, but is not limited thereto. In some embodiments, the cervical navigator 110 is constructed of hard plastic, silicone, and/or metal. That is, unlike other embodiments, the cervical navigator 110 does not deform (e.g., expand, enlarge). In some embodiments, the cervical navigator 110 includes a first portion 111, a second portion 112, a third portion 113, and a thumb recess 114. Collectively, the first portion 111, the second portion 112, and the third portion is referred to as an elongate body 111, 112, 113. The elongate body 111, 112, 113 is constructed to have a circumference large enough to receive the largest size dilator of the plurality of dilators 130, such as, for example, a dilator having a circumference and/or a diameter greater than each other dilator of the plurality of dilators 130. The thumb recess 114 is disposed on at least a portion of the third portion 113. In some embodiments, the retractor 120 includes a handle 121, a plurality of finger recesses 122, and a sheath-receiving surface 123, but is not limited thereto. The handle 121 has an arcuate and/or curved structure. In particular, the handle 121 facilitates gripping thereof. The plurality of finger recesses 122 are disposed on at least a portion of the handle 121. Accordingly, the plurality of finger recesses 122 and the thumb recess 114 facilitate gripping thereof. For example, each of the plurality of finger recesses 122 receives at least one finger (e.g., a pointer finger, a middle finger, a ring finger, a pinky) thereon, and the thumb recess 114 receives a thumb thereon. As such, the plurality of finger recesses 122 and the thumb recess 114 facilitate movement of the cervical navigator 110 and the retractor 120 during a cervical dilation procedure. Also, the plurality of finger recesses 122 and the thumb recess 114 prevent accidental movement, such as slipping while at least one finger and the thumb are placed thereon. In some embodiments, the plurality of finger recesses 122 and the thumb recess 114 includes a friction-increasing surface, such as, for example, a rubber layer, an adhesive, a textured surface, a knurled surface, and the like.

In some embodiments, the sheath-receiving surface 123 is elongate and has a curved, semi-circular structure. The sheath-receiving surface 123 receives the elongate body 111, 112, 113 thereon. The sheath-receiving surface 123 facilitates insertion of at least the first portion 111 and the second portion 112 into the body cavity during the cervical dilation procedure. Specifically, the sheath-receiving surface 123 prevents efflux of a uterine distending medium. Furthermore, the retractor 120 retracts the posterior vaginal wall and access to the cervix without repetitive insertion and removal of a speculum. As such, the retractor 120 reduces trauma experienced by the patient during the cervical dilation procedure.

In the depicted example of FIG. 7B, in some embodiments, the cervical navigator 110 further includes a plurality of connector anchors 117 but is not limited thereto. Moreover, in some embodiments, the retractor 120 further includes a catheter channel 124 and a plurality of connector grooves 125 but is not limited thereto. In some embodiments, the catheter channel 124 is disposed within at least a portion of the retractor 120, such as, for example, the sheath-receiving surface 123. However, in different embodiments, the catheter channel 124 is disposed within the handle 121 and extends through the sheath-receiving surface 123. The catheter channel 124 is hollow and receives a tubular instrument, such as a catheter therein. The catheter channel 124 extends from a portion of the sheath-receiving surface 123 toward an end of the sheath-receiving surface 123 at a distance furthest (i.e., distal) from the handle 121. In other words, the catheter channel 124 extends a length of the retractor 120 and exits into a patient's vagina during an operation, so the operator can access an inflation unit (not shown). In some embodiments, the catheter is connected within a portion of the sheath-receiving surface 123 of the retractor 120. In different embodiments, the catheter is a separate device inserted into the catheter channel after dilation of the cervix.

The plurality of connector anchors 117 are disposed on at least a portion of the cervical navigator 110. The plurality of connector anchors 117 receive the retractor 120 therein and/or thereon. Stated differently, the retractor 120 removably connects and/or interconnects to the cervical navigator 110 via the plurality of connector anchors 117.

In some embodiments, the retractor 120 further includes a plurality of connector grooves 125. The plurality of connector grooves 125 correspond to the plurality of connector anchors 117. That is, in some embodiments, a number of the plurality of connector grooves 125 matches a number of the plurality of connector anchors 117. Therefore, the plurality of connector anchors 117 move (i.e., slide within) and interconnect to the plurality of connector grooves 125. In other words, the plurality of connector anchors 117 are removably connected (i.e., inserted) into the plurality of connector grooves 125 to connect the cervical navigator 110 to the retractor 120. Moreover, the plurality of anchors 117 prevent removal of the cervical navigator 110 from the retractor 120 while the plurality of anchors 117 remain connected (i.e., inserted) within the plurality of connector grooves 125. Accordingly, connecting the plurality of connector anchors 117 to the plurality of connector grooves 125 prevents separation of the cervical navigator 110 from the retractor 120, and therefore, prevents inadvertent damage to the patient during the cervical dilation procedure. In some embodiments, the plurality of connector anchors 117 are curved and/or hook shaped to hook into the plurality of connector grooves 125. In different embodiments, each of the plurality of connector anchors 117 are a push fit, snap fit, or snugly fit, such that they have a size (e.g., length, width, depth) that corresponds to a size (e.g., length, width, depth) of each of the plurality of connector grooves 125 and snap into the plurality of connector anchors 117.

The lever unit 140 is disposed on at least a portion of the cervical navigator 110. In the depicted example of FIG. 7C, a lever for each of the plurality of dilators 130 protrudes through a portion of the lever unit 140. Moreover, a lever for each of the plurality of dilators 130 connects and/or interconnects with at least one of a plurality of grooves of the lever unit 140. Specifically, a lever of each of the plurality of dilators 130 moves from at least one first of the plurality of grooves in a first section (e.g., a bottom portion) of the lever unit 140 to at least one second of the plurality of grooves in a second section (e.g., a top portion) of the lever unit 140. Accordingly, moving the lever of each of the plurality of dilators moves a dilator within the cervical navigator 110 toward the first portion 111 in a first direction to be extracted from the first portion 111 and through the cervix during cervical dilation. Conversely, moving the lever of each of the plurality of dilators moves a dilator within the cervical navigator 110 toward the third portion 113 in a second direction to be retracted into the first portion 111. Stated differently, advancing the lever from the resting to the active position moves the corresponding dilator and returning the dilator from the active position to the resting position moves the dilator out of the cervix.

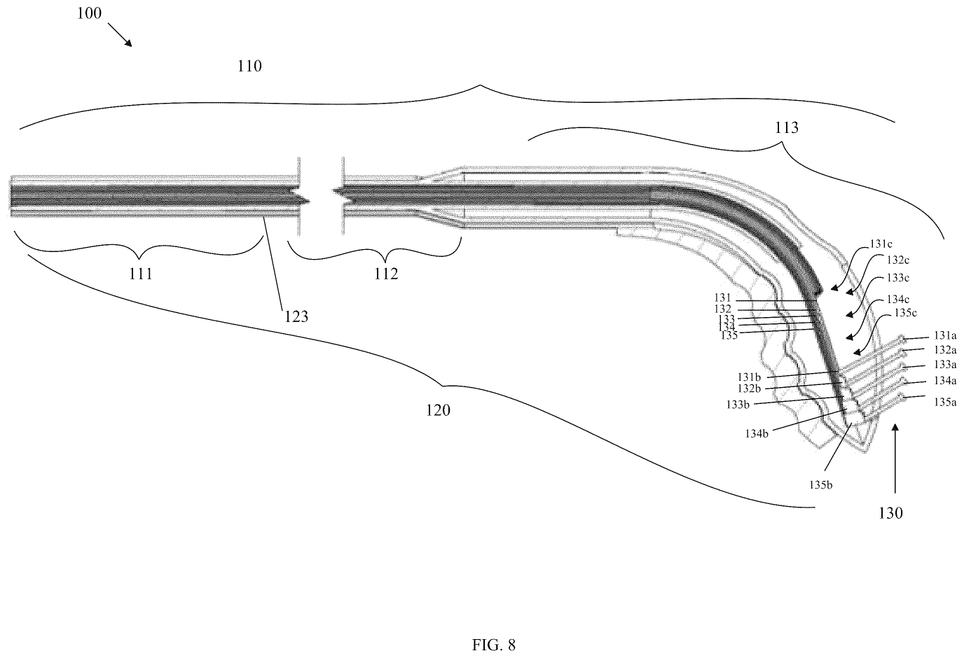

FIG. 8 is a cross-sectional view of the medical system 100 in accordance with some embodiments.

In the depicted example of FIG. 8, in some embodiments, the medical system 100 includes a cervical navigator 110, a retractor 120, and a plurality of dilators 130, but is not limited thereto. For sake of brevity, most components have been described above with respect to FIGS. 7A to 7C and will not be repeated here. In some embodiments, the plurality of dilators 130 includes a first dilator 131, a second dilator 132, a third dilator 133, a fourth dilator 134, and a fifth dilator 135, but is not limited thereto. In the depicted example of FIG. 8, there are five dilators. However, in different embodiments, there are less dilators or more dilators than in the depicted example. Also, each of the plurality of dilators 130 have different sizes which have been described above. It will be appreciated that use of the term lever herein also includes other means of control, such as, for example, a switch, a knob, a dial, a button, a touchscreen display, a slider, and the like.

Referring to FIG. 8, in some embodiments, the first dilator 131 includes a first dilator lever 131a, a first dilator connecting portion 131b, and a first dilator receiving recess 131c, but is not limited thereto. The first dilator 131 is movably (i.e., slidably) disposed within at least a portion of the cervical navigator 110 including the elongate body 111, 112, 113. The first dilator lever 131a is disposed at an end of the first dilator 131, preferably within the elongate body 111, 112, 113. The first dilator lever 131a protrudes from an interior of the elongate body 111, 112, 113 to a position outside a boundary (i.e., an exterior environment) of the elongate body 111, 112, 113. FIG. 8 depicts the position of the first dilator lever 131a protruding from the interior of the elongate body 111, 112, 113. The first dilator lever 131a facilitates gripping thereof. Furthermore, the first dilator 131 moves from a first position (i.e., proximal to an end of the first portion 111) through the elongate body 111, 112, 113 to at least partially toward a second position (i.e., proximal to an end of the elongate body 111, 112, 113, and more specifically, an end of the first portion 111 that is at a distance furthest from the end of the first portion 111). Conversely, the first dilator 131 moves from (i.e., can be withdrawn from) the second position back to the first position (e.g., retracting away from the end of the elongate body 111, 112, 113). To illustrate via an example, the first dilator lever 131a is moved in response to an application of force (e.g., pushing, pulling, sliding) to move the first dilator 131 through the cervical navigator 110 and dilate a body cavity, such as a cervix of a patient. This dilator can be withdrawn by returning the lever to the resting position, or left in place to facilitate advancement of the next sequential dilator.

The first dilator connecting portion 131b is disposed on at least a portion of the first dilator lever 131a that connects the first dilator lever 131a to the first dilator 131. Moreover, the first dilator connecting portion 131b removably connects the first dilator 131 to a connecting portion of at least one other dilator, such as, for example, the second dilator 132. It is important to note that moving the first dilator 131 from the first position to the second position moves the first dilator 131 away from a lever of the second dilator 132, while moving the first dilator 131 from the second position to the first position moves the first dilator 131 toward the lever of the second dilator 132.

Referring to FIG. 8, in some embodiments, the second dilator 132 includes a second dilator 132a, a second dilator connecting portion 132b, and a second dilator receiving recess 132c, but is not limited thereto. The second dilator 132 is movably (i.e., slidably) disposed within at least a portion of the cervical navigator 110 including the elongate body 111, 112, 113. The second dilator lever 132a is disposed at an end of the second dilator 132, preferably within the elongate body 111, 112, 113. The second dilator lever 132a protrudes from an interior of the retractor 120 to a position outside a boundary (i.e., an exterior environment) of the elongate body 111, 112, 113. FIG. 8 depicts the position of the second dilator lever 132a protruding from the interior of the elongate body 111, 112, 113. The second dilator lever 132a facilitates gripping thereof. Furthermore, the second dilator 132 moves from a first position (i.e., proximal to an end of the first portion 111) through the elongate body 111, 112, 113 to at least partially toward a second position (i.e., proximal to an end of the elongate body 111, 112, 113, and more specifically, an end of the first portion 111 that is at a distance furthest from the end of the first portion 111). Conversely, the second dilator 132 moves from the second position to the first position (e.g., retracting away from the end of the elongate body 111, 112, 113). To illustrate via an example, the second dilator lever 132a is moved in response to an application of force (e.g., pushing, pulling, sliding) to move the second dilator 132 through the cervical navigator 110 and dilate a body cavity, such as a cervix of a patient.

The second dilator connecting portion 132b is disposed on at least a portion of the second dilator lever 132a that connects the second dilator lever 132a to the second dilator 132. Moreover, the second dilator connecting portion 132b removably connects the second dilator 132 to a connecting portion of at least one other dilator, such as, for example, the first dilator 131 and/or the third dilator 133. Movement of the second dilator lever 132a, from the resting to the active position, advances the corresponding cervical dilator along the elongate body towards and ultimately through the cervix. The dilator can be left in the active position for the next dilator to cascade over, or withdrawn out of the cervix by moving the second dilator lever 132a back to the initial resting position. It is important to note that moving the second dilator 132 from the first position to the second position moves the second dilator 132 away from the first dilator lever 131a and/or the third dilator 133, while moving the second dilator 132 from the second position to the first position moves the second dilator 132 toward the first dilator lever 131a and/or the third dilator 133.

Referring to FIG. 8, in some embodiments, the third dilator 133 includes a third dilator 133a, a third dilator connecting portion 133b, and a third dilator receiving recess 133c, but is not limited thereto. The third dilator 133 is movably (i.e., slidably) disposed within at least a portion of the cervical navigator 110 including the elongate body 111, 112, 113. The third dilator lever 133a is disposed at an end of the third dilator 133, preferably within the elongate body 111, 112, 113. The third dilator lever 133a protrudes from an interior of the elongate body 111, 112, 113 to a position outside a boundary (i.e., an exterior environment) of the retractor 120. FIG. 8 depicts the position of the third dilator lever 133a protruding from the interior of the elongate body 111, 112, 113. The third dilator lever 133a facilitates gripping thereof. Furthermore, the third dilator 133 moves from a first position (i.e., proximal to an end of the first portion 111) through the elongate body 111, 112, 113 to at least partially toward a second position (i.e., proximal to an end of the elongate body 111, 112, 113, and more specifically, an end of the first portion 111 that is at a distance furthest from the end of the first portion 111). Conversely, the third dilator 133 moves from the second position to the first position (e.g., retracting away from the end of the elongate body 111, 112, 113). To illustrate via an example, the third dilator lever 133a is moved in response to an application of force (e.g., pushing, pulling, sliding) to move the third dilator 133 through the outer sheath 110 to increase the size of the cervical navigator 110 and dilate a body cavity, such as a cervix of a patient.

The third dilator connecting portion 133b is disposed on at least a portion of the third dilator lever 133a that connects the third dilator lever 133a to the third dilator 133. Moreover, the third dilator connecting portion 133b removably connects the third dilator 133 to a connecting portion of at least one other dilator, such as, for example, the second dilator 132 and/or the fourth dilator 134. Movement of the third dilator lever 133a, from the resting to the active position, advances the corresponding cervical dilator along the elongate body towards and ultimately through the cervix. The dilator can be left in the active position for the next dilator to cascade over, or withdrawn out of the cervix by moving the third dilator lever 133a back to the initial resting position. It is important to note that moving the third dilator 133 from the first position to the second position moves the third dilator 133 away from the second dilator lever 132a and/or the fourth dilator 134, while moving the third dilator 133 from the second position to the first position moves the third dilator 133 toward the second dilator lever 132a and/or the fourth dilator 134.

Referring to FIG. 8, in some embodiments, the fourth dilator 134 includes a fourth dilator 134a, a fourth dilator connecting portion 134b, and a fourth dilator receiving recess 134c, but is not limited thereto. The fourth dilator 134 is movably (i.e., slidably) disposed within at least a portion of the cervical navigator 110 including the elongate body 111, 112, 113. The fourth dilator lever 134a is disposed at an end of the fourth dilator 134, preferably within the elongate body 111, 112, 113. The fourth dilator lever 134a protrudes from an interior of the elongate body 111, 112, 113 to a position outside a boundary (i.e., an exterior environment) of the elongate body 111, 112, 113. FIG. 8 depicts the position of the fourth dilator lever 134a protruding from the interior of the retractor 120. The fourth dilator lever 134a facilitates gripping thereof. Furthermore, the fourth dilator 134 moves from a first position (i.e., proximal to an end of the first portion 111) through the elongate body 111, 112, 113 to at least partially toward a second position (i.e., proximal to an end of the elongate body 111, 112, 113, and more specifically, an end of the first portion 111 that is at a distance furthest from the end of the first portion 111). Conversely, the fourth dilator 134 moves from the second position to the first position (e.g., retracting away from the end of the elongate body 111, 112, 113). To illustrate via an example, the fourth dilator lever 134a is moved in response to an application of force (e.g., pushing, pulling, sliding) to move the fourth dilator 134 through the cervical navigator 110 and dilate a body cavity, such as a cervix of a patient.

The fourth dilator connecting portion 134b is disposed on at least a portion of the fourth dilator lever 134a that connects the fourth dilator lever 134a to the fourth dilator 134. Moreover, the fourth dilator connecting portion 134b removably connects the fourth dilator 134 to a connecting portion of at least one other dilator, such as, for example, the third dilator 133 and/or the fifth dilator 135. Movement of the fourth dilator lever 134a, from the resting to the active position, advances the corresponding cervical dilator along the elongate body towards and ultimately through the cervix. The dilator can be left in the active position for the next dilator to cascade over, or withdrawn out of the cervix by moving the fourth dilator lever 134a back to the initial resting position. It is important to note that moving the fourth dilator 134 from the first position to the second position moves the fourth dilator 134 away from the third dilator lever 133a and/or the fifth dilator 135, while moving the fourth dilator 134 from the second position to the first position moves the fourth dilator 134 toward the third dilator lever 133a and/or the fifth dilator 135.

Referring to FIG. 8, in some embodiments, the fifth dilator 135 includes a fifth dilator 135a, a fifth dilator connecting portion 135b, and a fifth dilator receiving recess 135c, but is not limited thereto. The fifth dilator 135 is movably (i.e., slidably) disposed within at least a portion of the cervical navigator 110 including the elongate body 111, 112, 113. The fifth dilator lever 135a is disposed at an end of the fifth dilator 135, preferably within the elongate body 111, 112, 113. The fifth dilator lever 135a protrudes from an interior of the elongate body 111, 112, 113 to a position outside a boundary (i.e., an exterior environment) of the elongate body 111, 112, 113. FIG. 8 depicts the position of the fifth dilator lever 135a protruding from the interior of the elongate body 111, 112, 113. The fifth dilator lever 135a facilitates gripping thereof. Furthermore, the fifth dilator 135 moves from a first position (i.e., proximal to an end of the first portion 111) through the elongate body 111, 112, 113 to at least partially toward a second position (i.e., proximal to an end of the elongate body 111, 112, 113, and more specifically, an end of the first portion 111 that is at a distance furthest from the end of the first portion 111). Conversely, the fifth dilator 135 moves from the second position to the first position (e.g., retracting away from the end of the elongate body 111, 112, 113). To illustrate via an example, the fifth dilator lever 135a is moved in response to an application of force (e.g., pushing, pulling) to move the fifth dilator 135 through the cervical navigator 110 and dilate a body cavity, such as a cervix of a patient.

The fifth dilator connecting portion 135b is disposed on at least a portion of the fifth dilator lever 135a that connects the fifth dilator lever 135a to the fifth dilator 135. Moreover, the fifth dilator connecting portion 135b removably connects the fifth dilator 135 to a connecting portion of at least one other dilator, such as, for example, the fourth dilator 135. Movement of the fifth dilator lever 135a, from the resting to the active position, advances the corresponding cervical dilator along the elongate body towards and ultimately through the cervix. The dilator can be left in the active position for the next dilator to cascade over, or withdrawn out of the cervix by moving the fifth dilator lever 135a back to the initial resting position. It is important to note that moving the fifth dilator 135 from the first position to the second position moves the fifth dilator 135 away from the fourth dilator lever 134a, while moving the fifth dilator 135 from the second position to the first position moves the fifth dilator 135 toward the fourth dilator lever 134a.

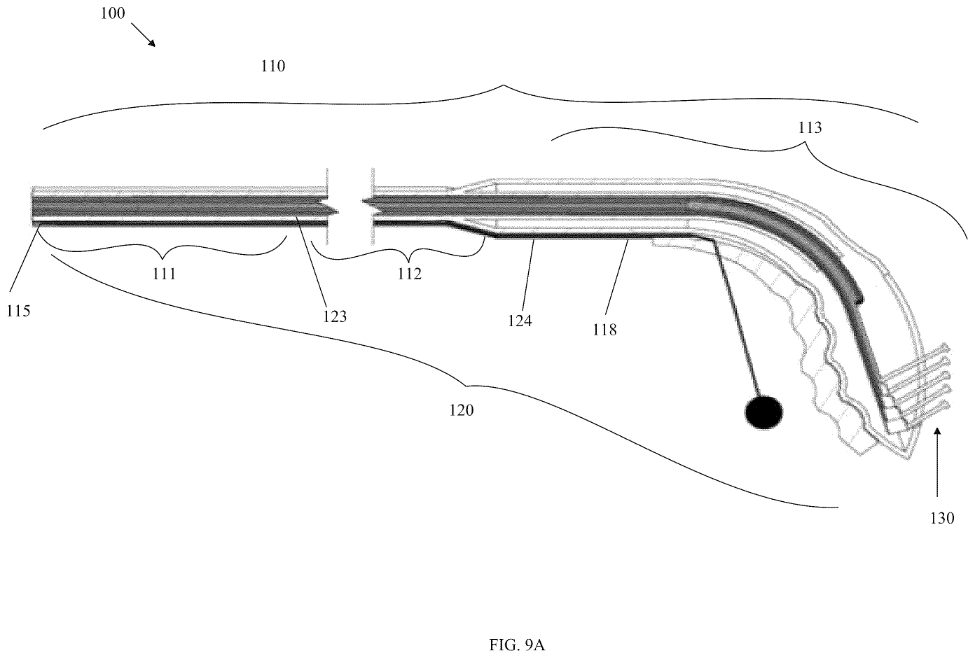

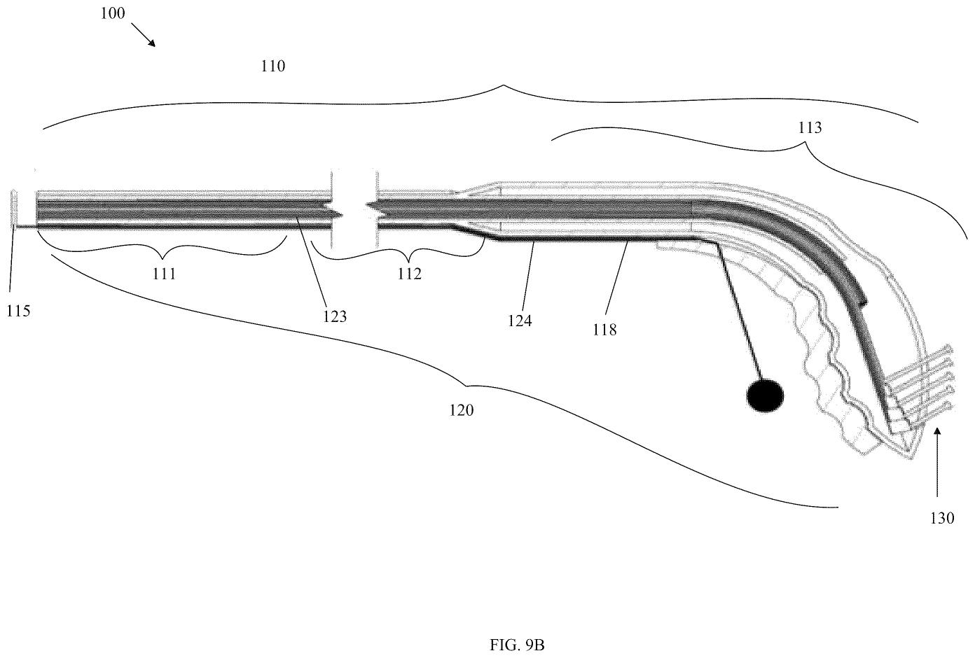

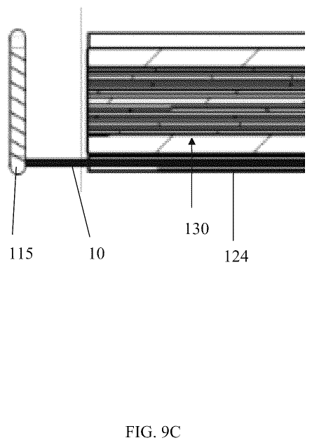

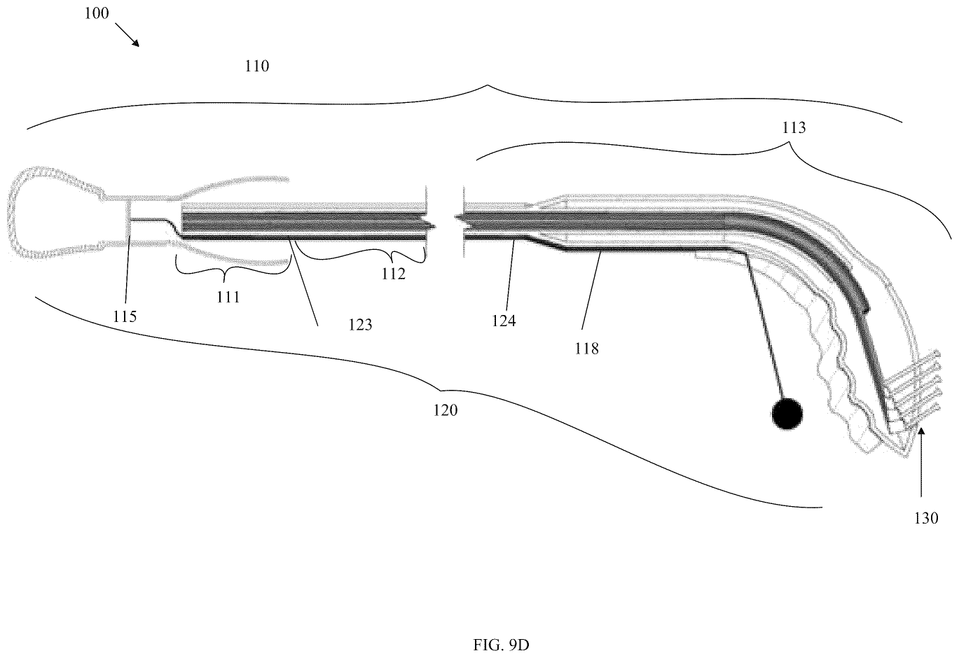

FIG. 9A is a cross-sectional view of the medical system 100 including an inflation unit 127 as disposed in the retractor 120 in accordance with some embodiments.

FIG. 9B is a cross-sectional side view of the medical system 100 after inflating the inflation unit 127 in accordance with some embodiments.

FIG. 9C is a zoomed-in cross-sectional side view of the inflation unit 127 after inflation in accordance with some embodiments.

FIG. 9D is a cross-sectional side view of the medical system 100 after inflating the inflation unit 127 as disposed in a cervix in accordance with some embodiments.

In the depicted example of FIGS. 9A through 9D, in some embodiments, the medical system 100 includes a cervical navigator 110, a retractor 120, and a plurality of dilators 130, but is not limited thereto. For sake of brevity, most components have been described above with respect to FIGS. 7A to 7C and will not be repeated here. In some embodiments, the retractor 120 further includes an inflation unit 127 and a catheter 128 connected to the inflation unit 127. In contrast to the embodiments of FIGS. 1-6, in the depicted examples of FIGS. 9A through 9D, the inflation unit 127 is disposed at an end of the sheath-receiving surface 123. Moreover, in some embodiments, the inflation unit 127 is ring-shaped. That is, the inflation unit 127 has a hole at a center portion thereof. The inflation unit 127 inflates at a predetermined distance away from the end of the sheath-receiving surface 123. For example, the inflation unit 127 is inflated within a range of two to six centimeters (cm) from the end of the sheath-receiving surface 123 that is based on movement of the catheter 128 to extend the inflation unit 127 into the cervix of the patient. In some embodiments, the catheter 128 is disposed within the catheter channel 124. However, in different embodiments, the catheter 128 is a separate unit and not integrated with or disposed within the retractor 120.

Referring to FIG. 9A, the inflation unit 127 is not yet inflated. However, in FIG. 9B and 9C, the inflation unit 127 has been inflated in response to receiving liquid or air through the catheter 128. For example, depressing a button, squeezing the button, and/or connecting a syringe that dispenses the liquid or the air through the catheter 128 to inflate the inflation unit 127. As described above, in the cervical dilation procedure, the cervical navigator 110 and the retractor 120 are inserted into the body cavity (e.g., vagina, cervix, uterus). After the cervical navigator 110, and in particular, the first portion 111 contacts (e.g., abuts) the cervix, at least one of the plurality of dilators 130 is inserted through the cervical navigator 110 using one or more of the levers of at least one of the plurality of dilators 130. The amount of dilation varies based on the needs of the patient and type of procedure. After the cervix has been dilated as needed based on at least one of the plurality of dilators 130 inserted into the cervix, the catheter 128 is moved (i.e., pushed, pulled, glide, slide) through the catheter channel 124 to extend the inflation unit 127 into and through the cervix to reach the internal os. Referring to FIGS. 9B through 9D, the inflation unit 127 is inflated and then the cervical navigator 110 is extracted (e.g., removed, detached) from the retractor 120. Stated differently, referring again to FIG. 7B, the plurality of connector anchors 117 of the cervical navigator 110 are removed from the plurality of connector grooves 125 from the retractor 120, such that only the retractor 120 remains connected to the cervix and vagina (now shown). In this manner, the inflation unit 127 prevents the retractor 120 from moving (i.e., falling) off the body cavity and maintains access into the cervix and allows for traction on the cervix without the addition of additional devices. Additional operating instruments may be inserted through the inflation unit 127.

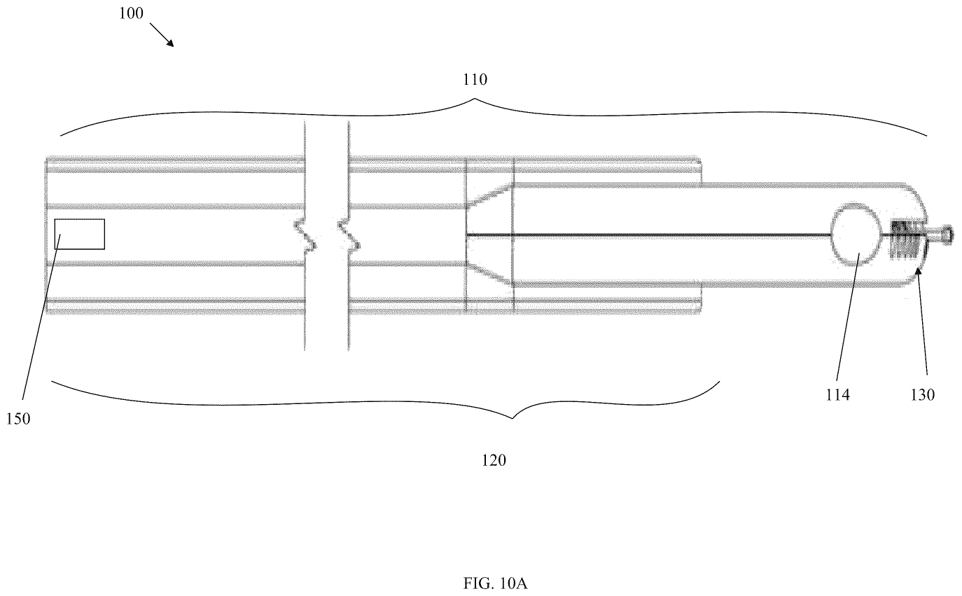

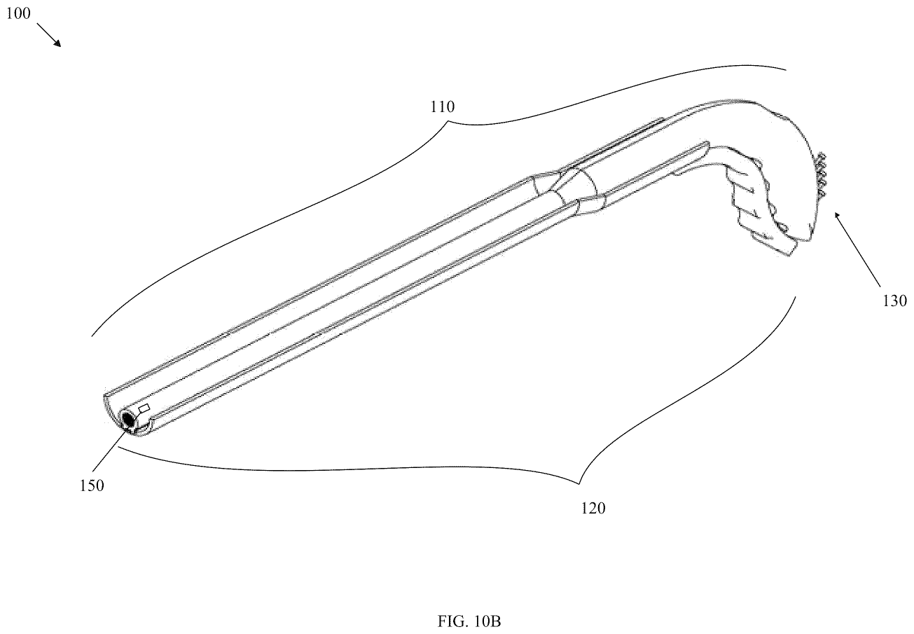

FIG. 10A is an elevational top view of the medical system 100 in accordance with some embodiments.

FIG. 10B is a perspective top view of the medical system 100 in accordance with some embodiments.

FIG. 10C is a perspective top view of the medical system 100 after inflating the inflation unit 127 in accordance with some embodiments.

In the depicted example of FIGS. 10A through 10C, in some embodiments, the medical system 100 includes a cervical navigator 110, a retractor 120, and a plurality of dilators 130, but is not limited thereto. For sake of brevity, most components have been described above with respect to FIGS. 7A to 7C and will not be repeated here. In some embodiments, the cervical navigator device 100 further includes a light 150, but is not limited thereto. In the depicted examples of FIGS. 10A through 10C, the light 150 is removably disposed (i.e., detachable) on at least a portion of the cervical navigator 110 or the retractor 120. In other embodiments, the light 150 is permanently disposed (i.e., attached) on the cervical navigator 110 or the retractor 120. More specifically, the light 150 is disposed at an end of and/or within the retractor 120. However, in different embodiments, the light 150 is disposed on at least a portion of a first side (e.g., left), a second side (e.g., right), and/or a bottom of the retractor 120. Alternatively, and/or in addition thereto, the light 150 is disposed on each side of the retractor 120 proximal to the end of the retractor 120. Furthermore, the light 150 is connected to a switch or a button with a power source (e.g., a battery, a solar cell). The light 150 illuminates the cervical navigator 110, the retractor 120, and/or an area surrounding the cervical navigator 110 and/or the retractor 120, such as, for example a vaginal cavity and improve visualization of the cervix. Accordingly, the light 150 improves an illumination level to facilitate viewing of the vagina and cervix during the cervical dilation procedure.

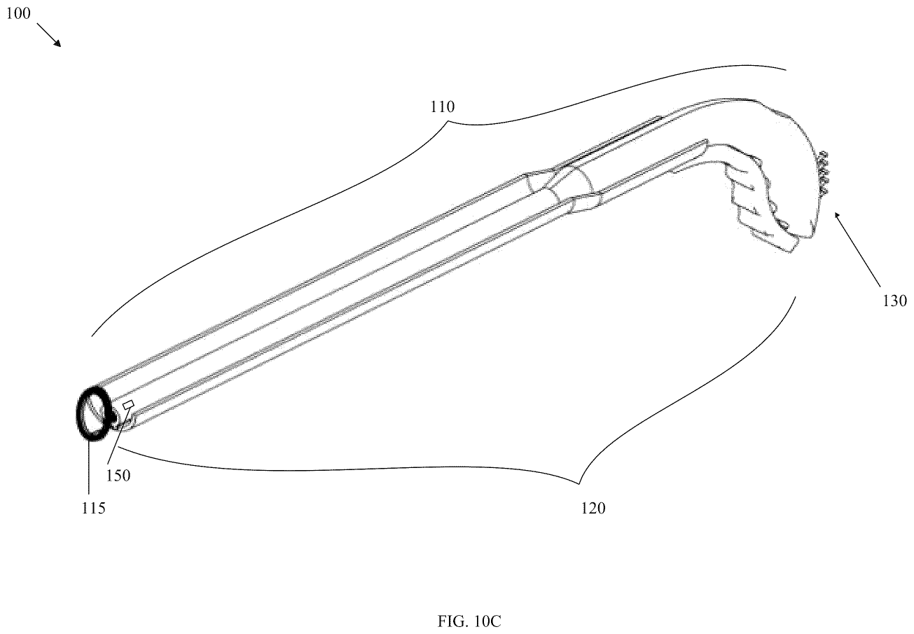

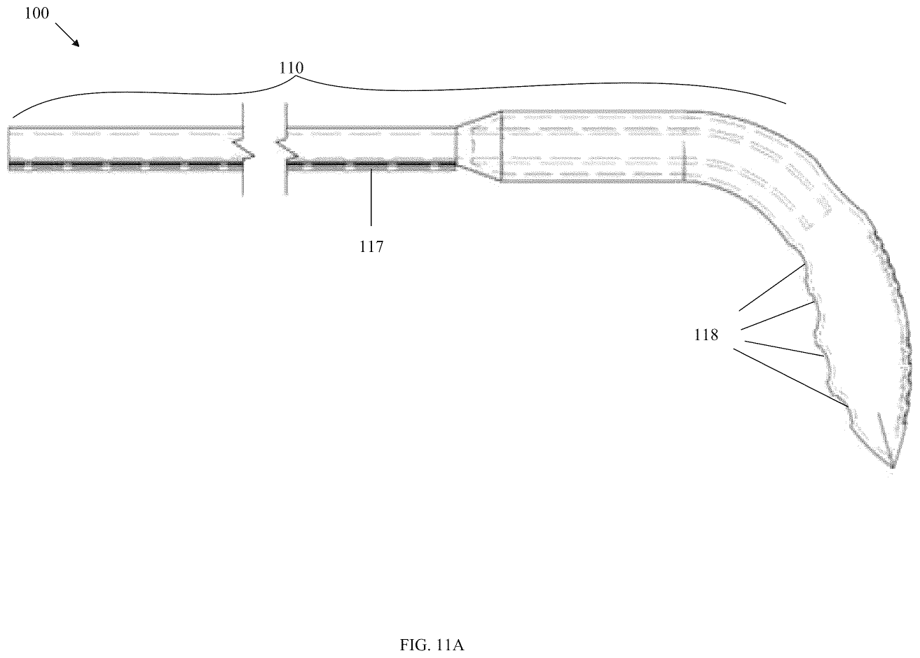

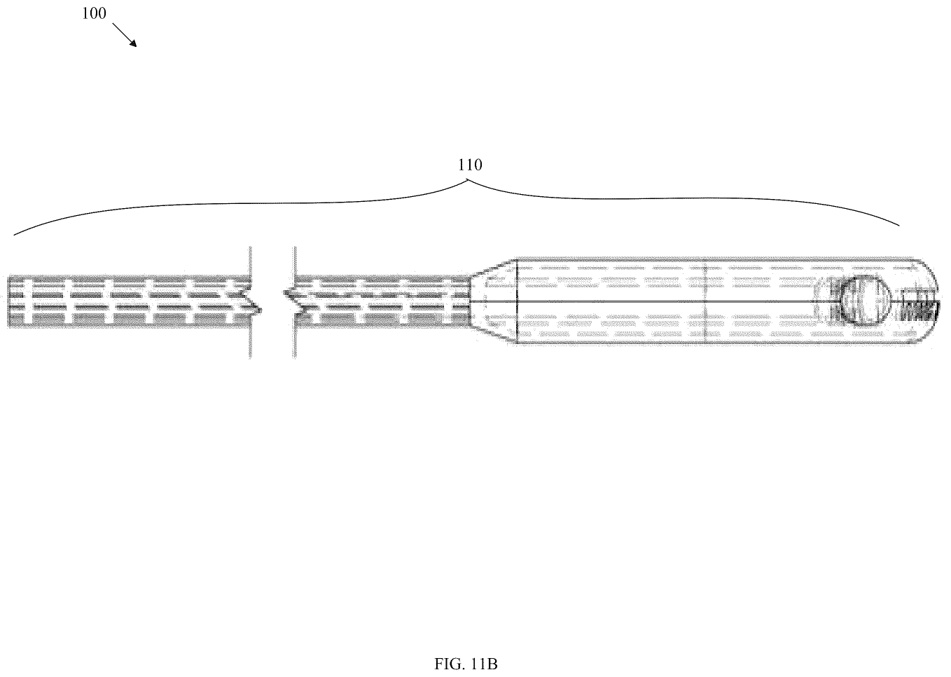

FIG. 11A is a cross-sectional side view of a cervical navigator 110 in accordance with some embodiments.

FIG. 11B is a cross-sectional top view of the cervical navigator 110 in accordance with some embodiments

FIG. 11C is a perspective top view of the cervical navigator 110 in accordance with some embodiments.

In the depicted example of FIGS. 11A through 11C, in some embodiments, the cervical navigator 110 includes the plurality of connector anchors 117. For sake of brevity, most components have been described above with respect to FIGS. 7A through 7C and will not be repeated here. In the depicted example of FIGS. 11A through 11C, the cervical navigator 110 further includes a plurality of recesses 118, but is not limited thereto. The plurality of recesses 118 are disposed on at least a portion of the elongate body 111, 112, 113. More specifically, the plurality of recesses 118 are disposed on at least a portion of the third portion 113. The plurality of recesses 118 are recessed and/or concave with respect to a surrounding surface of the third portion 113. The plurality of recesses 118 may facilitate gripping thereof.

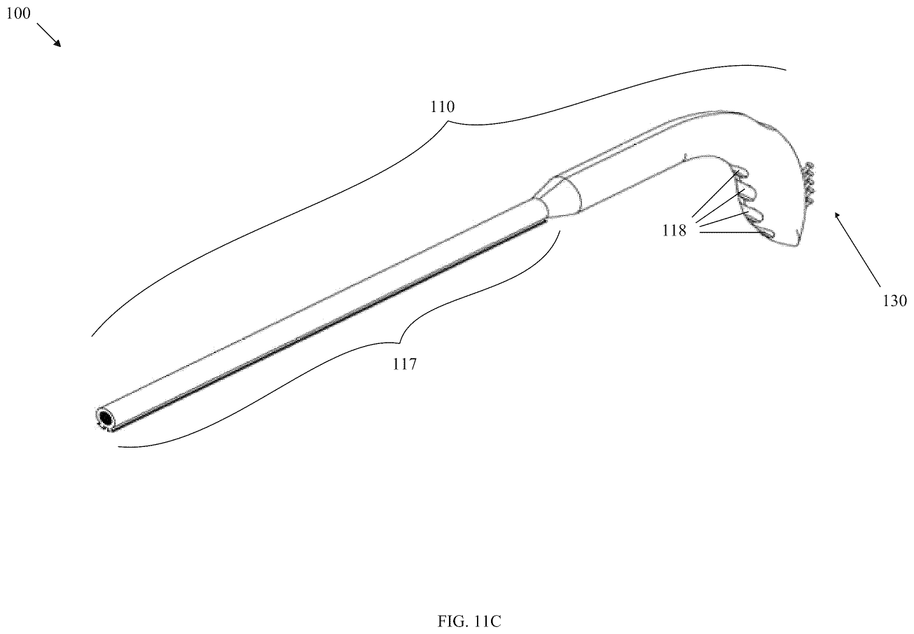

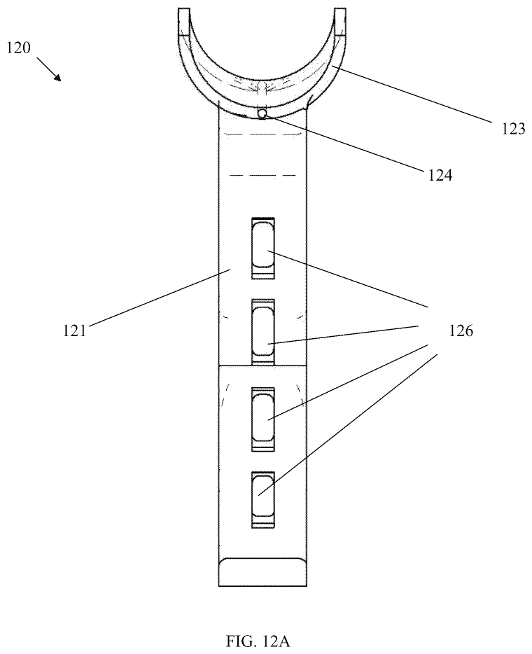

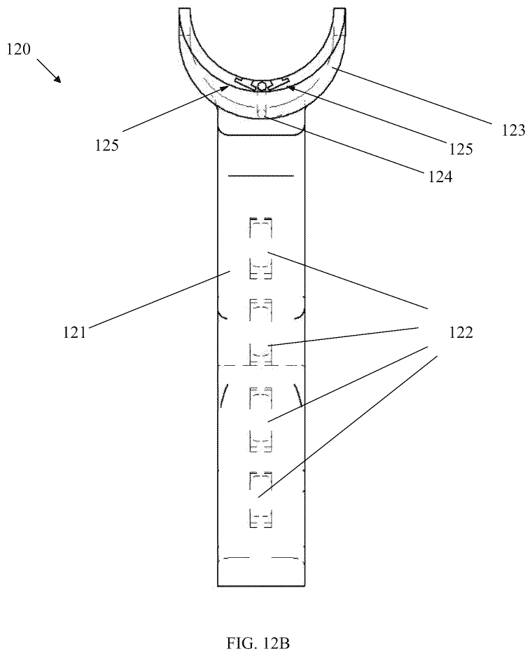

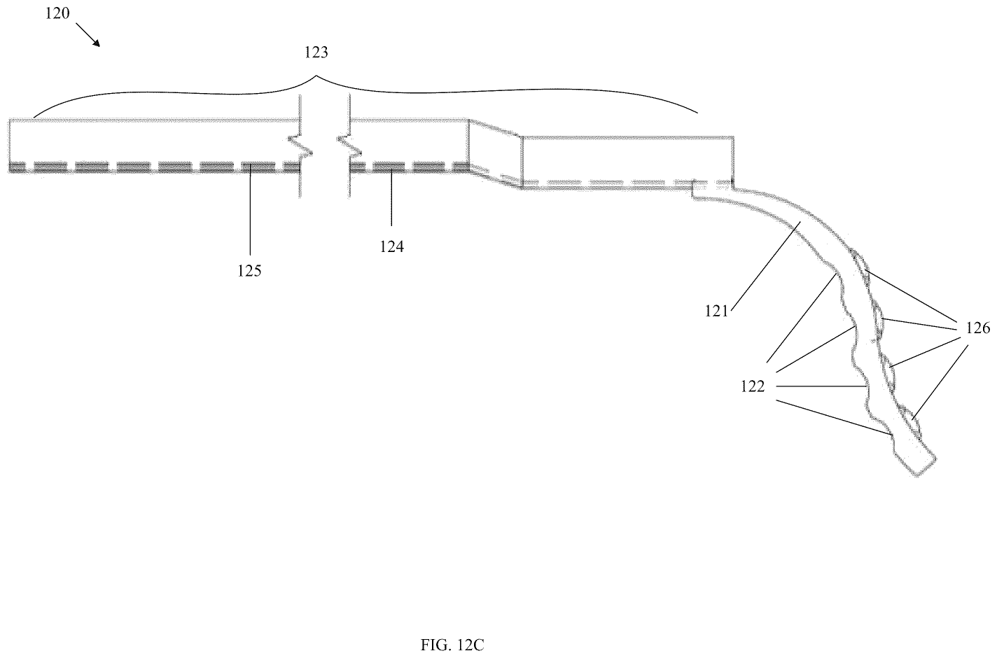

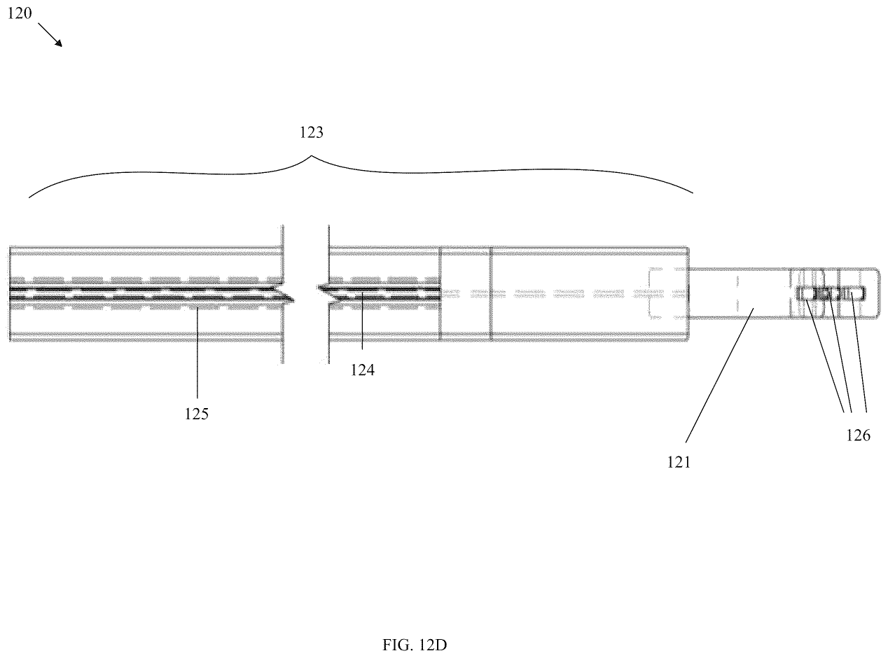

FIG. 12A is an elevational rear view of a posterior vaginal wall retractor 120 in accordance with some embodiments.

FIG. 12B is an elevational front view of the posterior vaginal wall retractor 120 in accordance with some embodiments.

FIG. 12C is a cross-sectional side view of the posterior vaginal wall retractor 120 in accordance with some embodiments.

FIG. 12D is an elevational top view of the posterior vaginal wall retractor 120 in accordance with some embodiments.

In the depicted example of FIGS. 12A through 12D, in some embodiments, the retractor 120 includes a handle 121, a plurality of finger recesses 122, a sheath-receiving surface 123, a catheter channel 124, and a plurality of connector grooves 125, but is not limited thereto. For sake of brevity, most components have been described above with respect to FIGS. 7A through 7C and will not be repeated here. Referring to FIGS. 12A, 12C, and 12D, in some embodiments, the retractor 120 further includes a plurality of connector protrusions 126, but is not limited thereto. The plurality of connector protrusions 126 are disposed on at least a portion of the handle 121. In particular, the plurality of finger recesses 122 are disposed on at least a portion of a first side of the handle 121 and the plurality of connector protrusions 126 are disposed on at least a portion of a second side of the handle 121 opposite with respect to the first side of the handle 121. The plurality of connector protrusions 126 are removably connected (i.e., inserted) within the plurality of recesses 118 on the third portion 113 of the cervical navigator 110. As such, the plurality of connector protrusions 126 facilitate interconnection between the handle 121 and at least a portion of the third portion 113. It will be appreciated that a number (e.g., four) of the plurality of connector protrusions 126 correspond to a number (e.g., four) of the plurality of recesses 118. The plurality of recesses 118 facilitate connecting the cervical navigator 110 to the retractor 120 during the cervical dilation procedure. Subsequently, the plurality of recesses 118 and the plurality of connector protrusions 126 facilitate detachment of the cervical navigator 110 from the retractor 120 after completing the cervical dilation, and the retractor 120 can remain in the vagina anchored to the cervix after the inflation unit 127 has been inflated.

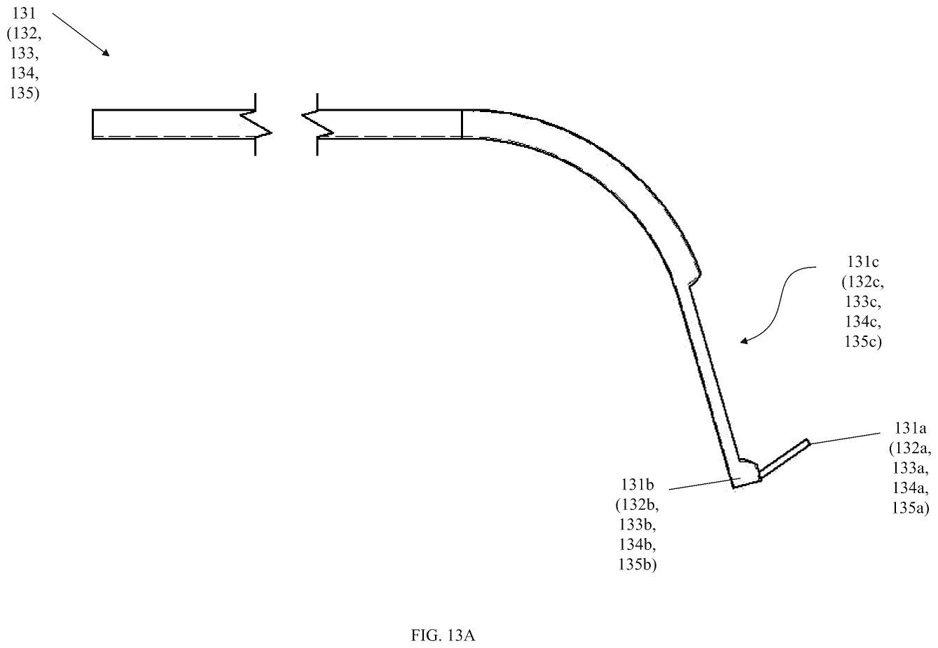

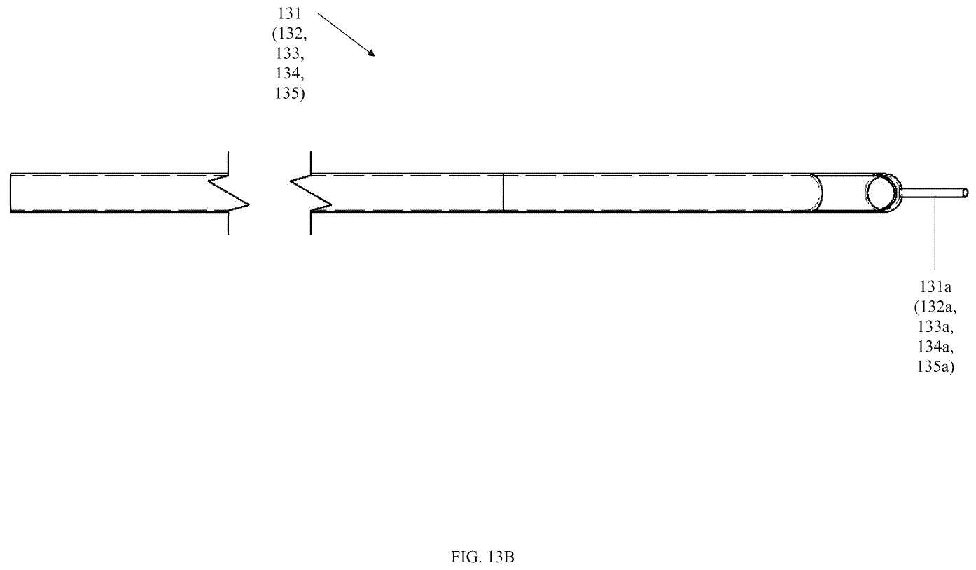

FIG. 13A is a side view of a dilator 131 in accordance with some embodiments.

FIG. 13B is a top view of a dilator 131 in accordance with some embodiments.

FIG. 13C is a rear view of a dilator 131 in accordance with some embodiments.

In the depicted example of FIGS. 13A to 13C, in some embodiments, the medical system 100 includes a plurality of dilators 130, but is not limited thereto. For sake of brevity, most components have been described above with respect to FIG. 8 and will not be repeated here.

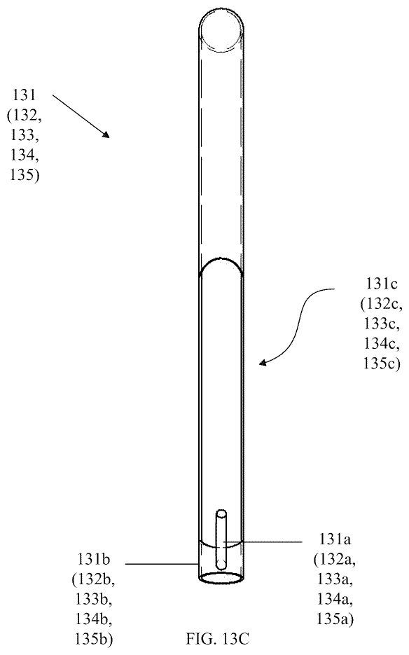

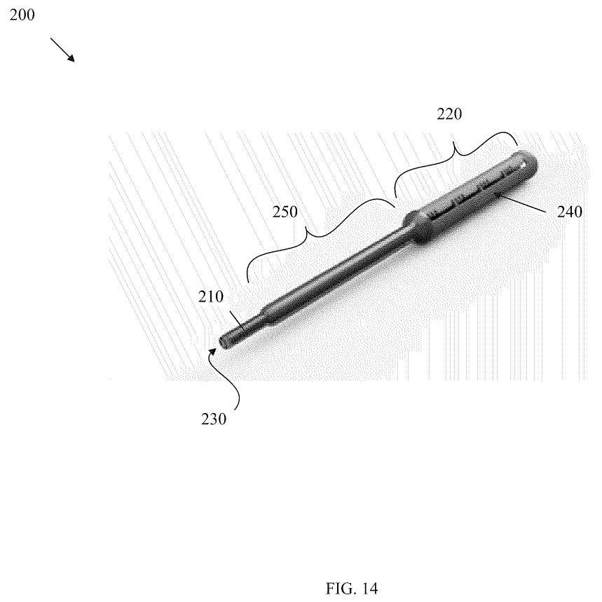

FIG. 14 is a perspective side view of a cervical navigator 200 in accordance with some embodiments.

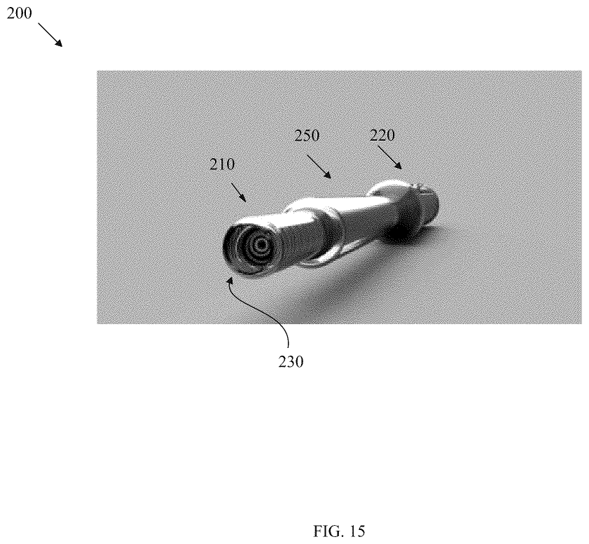

FIG. 15 is a perspective front view of the cervical navigator 200 in accordance with some embodiments.

In the depicted example of FIGS. 14 and 15, in some embodiments, the cervical navigator 200 includes an insertion tip 210, a handle 220, a plurality of dilators 230, a lever unit 240, and an outer sheath 250, but is not limited thereto. The insertion tip 210 is a relatively narrow and elongate structure. Moreover, the insertion tip 210 has a rigid structure in absence of an application of force (e.g., pushing, pulling, gliding, sliding). However, the insertion tip 210 at least partially deforms (e.g., bends, stretches, expands) in response to contact with living tissue (e.g., the vagina, the cervix, the uterus). The insertion tip 210 facilitates movement through living tissue, such as a body cavity. More specifically, the insertion tip 210 is inserted through the vagina and cervix during a cervical dilation procedure. The handle 220 is connected to the insertion tip 210 and facilitates gripping thereof. Referring to FIG. 15, the plurality of dilators 230 are sequentially cervical dilators nested within each other in a retracted (i.e., unextended) position.

The plurality of dilators 230 are movably (i.e., slidably) disposed within at least a portion of the insertion tip 210. In particular, each of the plurality of dilators 230 moves telescopically from an interior of the insertion tip 210 in a first position to at least partially toward an exterior away from the interior of the insertion tip. The lever unit 240 is disposed on at least a portion of the handle 220. The outer sheath 250 is disposed between the insertion tip 210 and the handle 220. The outer sheath 250 facilitates movement through living tissue, such as a body cavity.

FIG. 16 is a zoomed-in view of a lever unit 240 of the cervical navigator 200 in accordance with some embodiments.

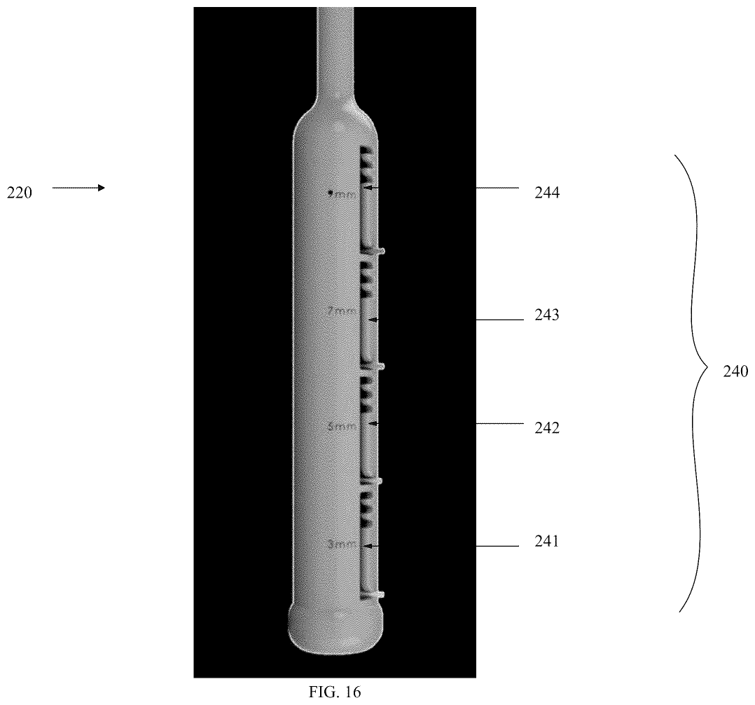

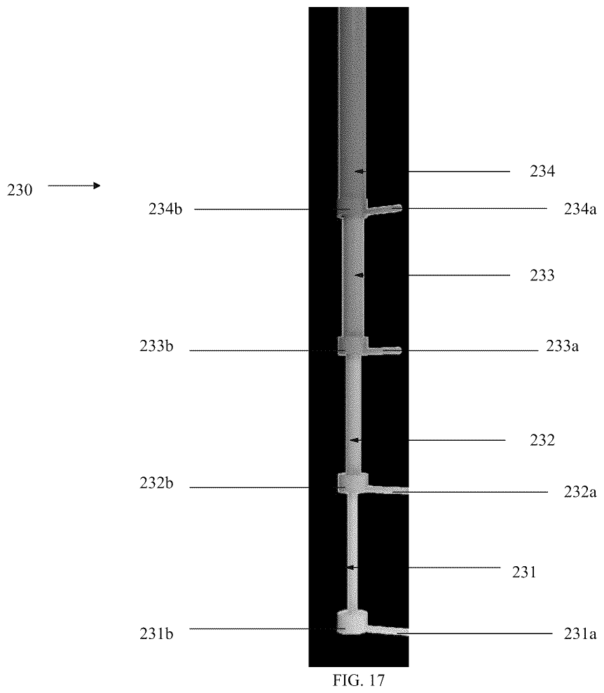

FIG. 17 is a zoomed-in view of a plurality of dilators 230 in accordance with some embodiments.

In some embodiments, the lever unit 240 includes a first lever section 241, a second lever section 242, a third lever section 243, and a fourth lever section 244, but is not limited thereto. In different embodiments, the lever unit 240 includes less sections or more sections than the four sections depicted in FIG. 16. In the depicted example of FIG. 16, the first lever section 241, the second lever section 242, the third lever section 243, and the fourth lever section 244 correspond to the plurality of dilators 230. In other words, each dilator of the plurality of dilators 230 moves within the respective lever section that matches a size (e.g., a diameter) of the dilator. For example, the dilator in the first section 241 manipulates a dilator with a 3 mm diameter. The dilator in the second section 242 manipulates a dilator with a 5 mm diameter. The dilator in the third section 243 manipulates a dilator with a 7 mm diameter. The dilator in the fourth section 244 manipulates a dilator with a 9 mm diameter.

In the depicted example of FIG. 17, in some embodiments, the plurality of dilators 230 includes a first dilator 231, a second dilator 232, a third dilator 233, and a fourth dilator 234, but is not limited thereto. In different embodiments, the plurality of dilators 230 includes less dilators or more dilators than the four dilators depicted in FIG. 17. For sake of brevity, each of the plurality of dilators 230 and associated components, are similar to the plurality of dilators 130 that have been described above with respect to FIG. 8 and will not be repeated here. Also, FIG. 17 depicts the plurality of dilators 230 nested within each other, in an unengaged position

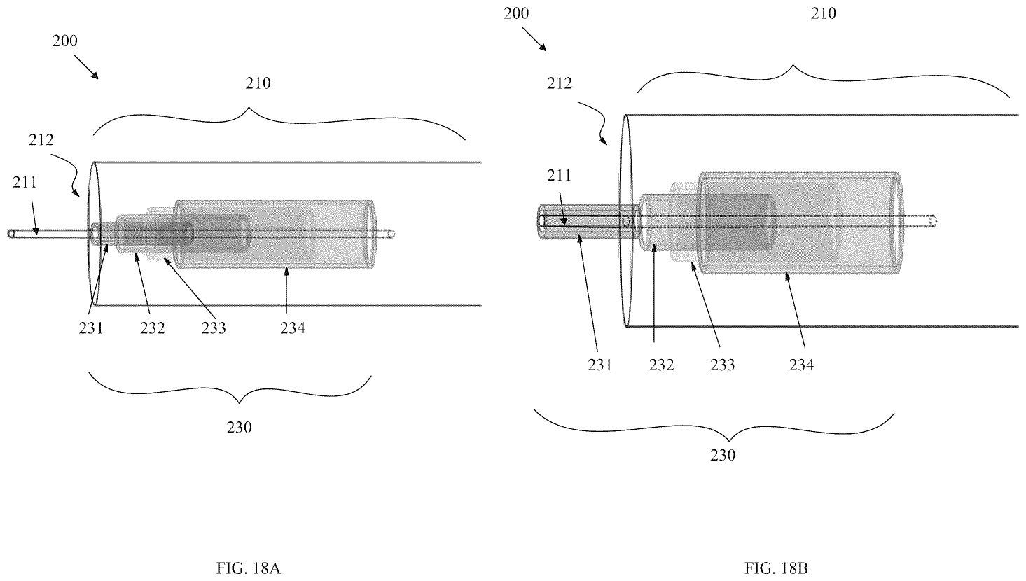

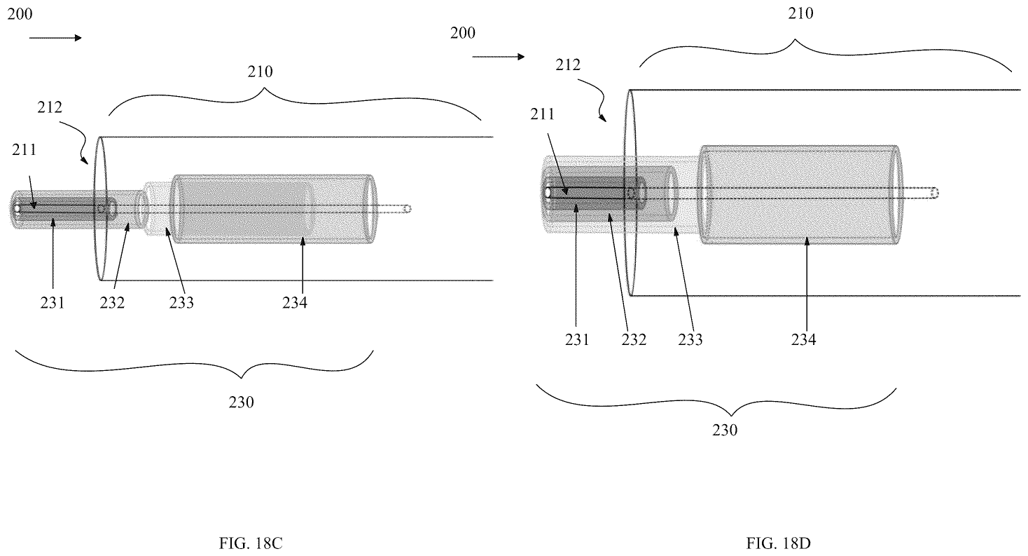

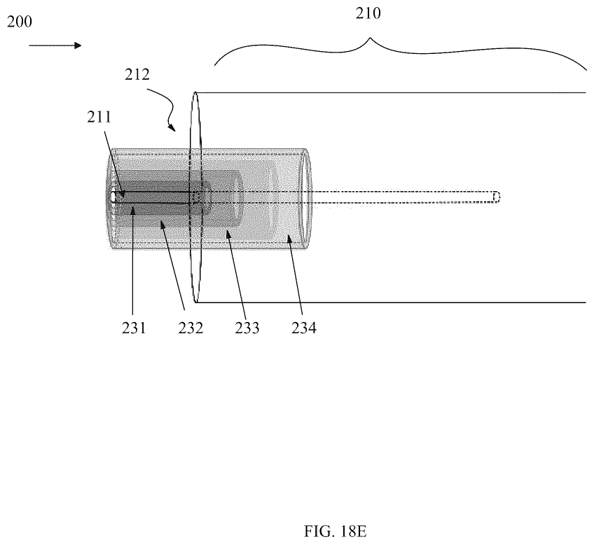

FIG. 18A is a cross-sectional side view of the plurality of dilators 230 in accordance with some embodiments. In the depicted example of FIG. 18A, in some embodiments, the insertion tip 210 includes an initial dilator 211 and an opening 212 but is not limited thereto. The initial dilator 211 is semi-rigid, elongate, and narrow. For example, in some embodiments, the initial dilator 211 has a 1 mm diameter. In different embodiments, the initial dilator 211 has a narrower or wider diameter than 1 mm. The initial dilator 211 is disposed at a center portion of the cervical navigator 200 within the insertion tip 210. Additionally, the initial dilator 211 extends from an interior portion of the insertion tip 210 through the opening 212 to a distance away from an end of the insertion tip 210. The initial dilator 211 is inserted into the body cavity (e.g., the vagina, cervix, and uterus) to facilitate use of the plurality of dilators 230 during a cervical dilation procedure. The initial dilator 211 at least partially deforms (e.g., bends, stretches, expands) in response to contact with living tissue (e.g., the vagina, the cervix, the uterus). Accordingly, the initial dilator 211 facilitates penetration of the body cavity and restricts movement of the plurality of dilators 230 to a length of the initial dilator 211. Each of the plurality of dilators is then sequentially advanced over the initial dilator 211 facilitating progressive cervical dilation without needing to re-navigate the cervix multiple times with each sequential dilator. In this embodiment the initial dilator 211 is shown first in its resting position and then in its fully advanced position having traversed the cervix for this initial passage. Each of the plurality of dilators 230 move (i.e., slide) over this initial dilator 211. The initial dilator 211 has a size (e.g., diameter, circumference) smaller than each of the plurality of dilators 230. Also, the initial dilator 211 is moved using a lever similar to levers of the plurality of dilators 230 further described below.

Referring to FIG. 18A, each of the plurality of dilators 230 is at least partially disposed within the insertion tip 210. In particular, in the depicted example of FIG. 18A, the plurality of dilators 230 are at least partially retracted within the insertion tip 210. In some embodiments, the plurality of dilators 230 are telescopic tubes that are each separately movable over other dilators. To illustrate via an example, the first dilator 231 is extended into the cervix. Thereafter, the second dilator 232 is extended is response to manipulation (e.g., pushing, pulling, rotating a knob, rotating a lever, etc.) of a second dilator lever 232a, such that the second dilator 232 telescopically extends over and surrounds the first dilator 231. Accordingly, the second dilator 232 has an opening and a channel at a center portion with a size (e.g., diameter, length) that corresponds to a size (e.g., diameter, length) of the first dilator 231. Specifically, the size of the opening and the channel of the second dilator 232 is greater than the size of the first dilator 231 to facilitate extending the second dilator 232 over and surrounding the first dilator 231.

FIG. 18B is a cross-sectional side view of the plurality of dilators 230 in accordance with some embodiments. In the depicted example of FIG. 18B, the first dilator 231 is extended over a length of the initial dilator 211 in response to movement of a first dilator lever 231a toward at least one groove of the lever unit 240. As such, the first dilator 231 expands (i.e., increases a size of) the cervix during the cervical dilation procedure. It will be appreciated that unlike some embodiments of the cervical navigator 110, the plurality of dilators 230 in the cervical navigator 200 protrude to the cervix to dilate the cervix, instead of increasing the size of the insertion tip 210.

FIG. 18C is a cross-sectional side view of the plurality of dilators 230 in accordance with some embodiments. In the depicted example of FIG. 18C, the second dilator 232 is extended over the length of the initial dilator 211, and/or to surround and cover (e.g., at least partially enclose) the first dilator 231 (from the configuration in FIG. 18B) in response to movement of a second dilator lever 232a toward at least one groove of the lever unit 240. As such, the second dilator 232 expands (i.e., increases a size of) the cervix during the cervical dilation procedure. It will be appreciated that the second dilator 232 has a size (e.g., diameter, circumference) greater than a size (e.g., diameter, circumference) of the first dilator 231. Accordingly, the second dilator 232 expands the cervix at a greater size than the first dilator 231.

FIG. 18D is a cross-sectional side view of the plurality of dilators 230 in accordance with some embodiments. In the depicted example of FIG. 18D, the third dilator 233 is extended over the length of the initial dilator 211, and/or to surround and cover (e.g., at least partially enclose) the second dilator 232 (from the configuration in FIG. 18C) in response to movement of a third dilator lever 233a toward at least one groove of the lever unit 240. As such, the third dilator 233 expands (i.e., increases a size of) the cervix during the cervical dilation procedure. It will be appreciated that the third dilator 233 has a size (e.g., diameter, circumference) greater than the size of the second dilator 232. Accordingly, the third dilator 233 expands the cervix at a greater size than the second dilator 232.

FIG. 18E is a cross-sectional side view of the plurality of dilators 230 in accordance with some embodiments. In the depicted example of FIG. 18E, the fourth dilator 234 is extended over the length of the initial dilator 211, and/or to surround and cover (e.g., at least partially enclose) the third dilator 233 (from the configuration in FIG. 18D) in response to movement of a fourth dilator lever 234a toward at least one groove of the lever unit 240. As such, the fourth dilator 234 expands (i.e., increases a size of) the cervix during the cervical dilation procedure. It will be appreciated that the fourth dilator 234 has a size (e.g., diameter, circumference) greater than the size of the third dilator 233. Accordingly, the fourth dilator 234 expands the cervix at a greater size than the third dilator 233.

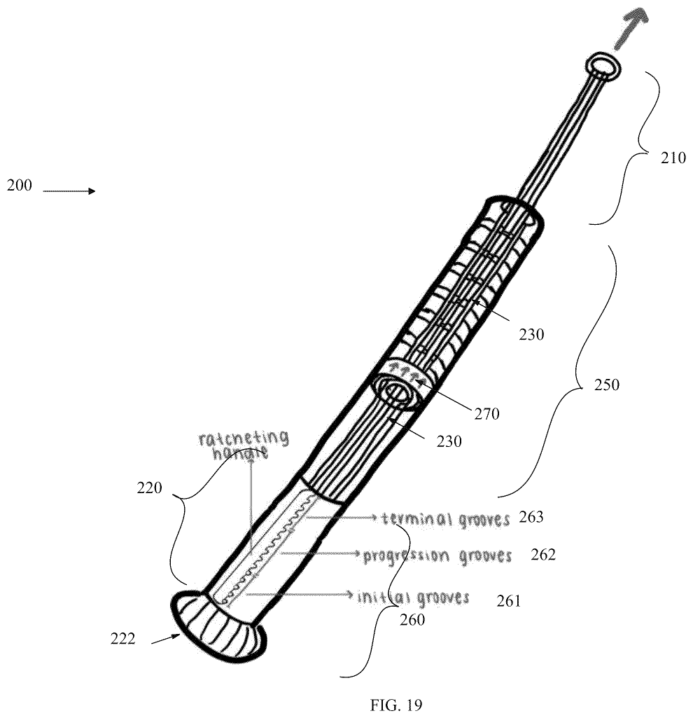

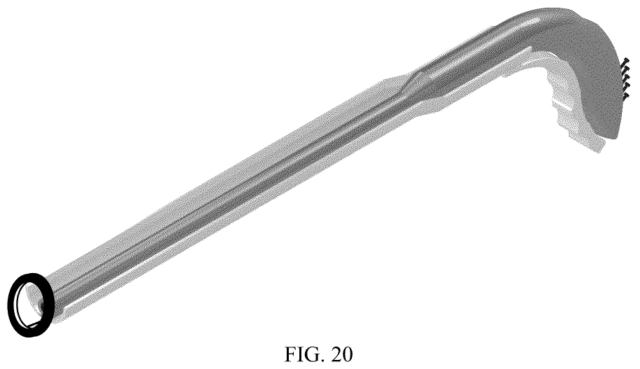

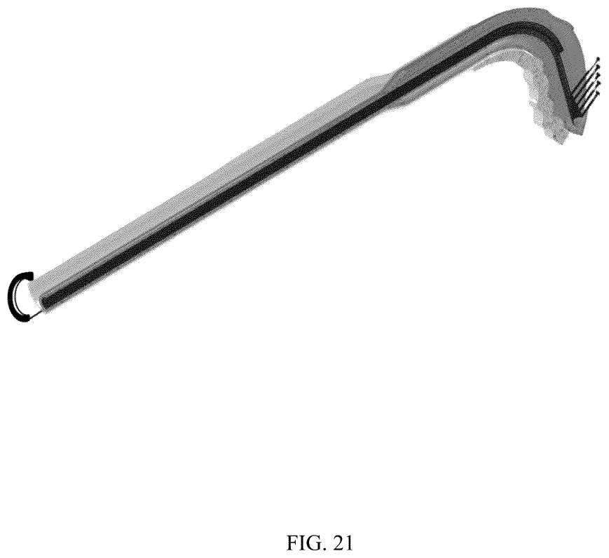

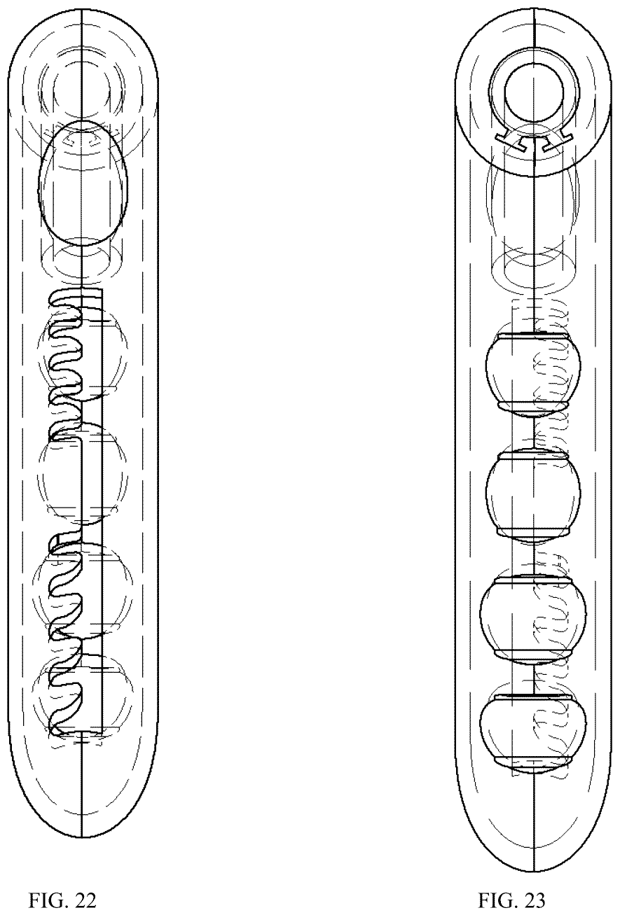

FIG. 19 is a cross-sectional side view of the lever unit 240 of the cervical navigator 200 in accordance with some embodiments. In the depicted example of FIG. 19, in some embodiments, the cervical navigator 200 further includes a lever track 260 and a slider 270, but is not limited thereto. In some embodiments, the lever track 260 includes a plurality of initial grooves 261, a plurality of progression grooves 262, and a plurality of terminal grooves 263, but is not limited thereto. Each of the plurality of initial grooves 261 is disposed at a first end of the lever track 260. The plurality of initial grooves 261 are connected to each of the plurality of dilators 230. At least one of the plurality of dilators 230 moves in a first direction toward the insertion tip 210 to be extracted from the insertion tip 210 in response to moving (i.e., rotating) a control knob 222 in a first rotating direction (i.e., clockwise). Conversely, at least one of the plurality of dilators 230 moves in a second direction opposite to the first direction within the insertion tip 210 to retract in the insertion tip 210 in response to moving the control knob 222 in a second rotating direction (i.e., counterclockwise). As such, each of the plurality of dilators 230 is controlled using the control knob 222. Moreover, the control knob 222 moves the slider 270 that is connected to the plurality of dilators 230. The slider 270 moves the plurality of dilators 230 in response to movement of the control knob 222. The plurality of initial grooves 261 set up the plurality of dilators 230 at a first predetermined distance within the insertion tip 210 while preparing for a cervical dilation procedure.

Each of the plurality of progression grooves 262 is disposed on at least a portion of the lever track 260, preferably between the plurality of initial grooves 261 and the plurality of terminal grooves 263. The plurality of progression grooves 262 are connected to each of the plurality of dilators 230. Based on control of the control knob 222, the plurality of dilators 230 are extracted from the insertion tip 210. After at least one of the plurality of dilators 230 is extracted to protrude from the insertion tip 210, the at least one dilator is connected to at least one of the pluralities of progression grooves 262. At this stage, the plurality of progression grooves 262 connect to the plurality of dilators 230 at a second predetermined distance within and during extraction from the insertion tip 210 for the cervical dilation procedure. It will be appreciated that the second predetermined distance is greater than the first predetermined distance with respect to a distance from the control knob 222.

Each of the plurality of terminal grooves 263 is disposed at a second end of the lever track 260. The plurality of terminal grooves 263 are connected to each of the plurality of dilators 230. Based on control of the control knob 222, the plurality of dilators 230 are extracted from the insertion tip 210. After at least one of the plurality of dilators 230 is extracted to protrude from the insertion tip 210 and is at a maximum distance from the control knob 222, the at least one dilator is connected to at least one of the plurality of terminal grooves 263. At this stage, the plurality of terminal grooves 263 connect to the plurality of dilators 230 at a third predetermined distance within and at full extraction from the insertion tip 210 for the cervical dilation procedure. It will be appreciated that the third predetermined distance is greater than the second predetermined distance with respect to a distance from the control knob 222.

In one embodiment, the cervical navigator device is also a tissue expanding device adapted for other medical uses other than just gynecological uses.

In one embodiment (FIGS. 1 and 2), the device includes an expandable outer sheath 110 that is passed into the cervix and secured with a balloon inflation unit 115 at the internal cervical ostia. The balloon is shaped like a donut with an open center to facilitate passage of instruments into the uterus while preventing egress of uterine distending medium. Sequential dilators of increasing size are then passed through the secured outer sheath until the desired cervical dilation is achieved. This allows the cervix to be dilated to the appropriate diameter while only having to navigate the cervical passageway once, reducing the risk of perforation or creation of false passages.

While in some embodiments, the outer sheath is an expandable material like soft plastic, rubber or silicone, in other embodiments the outer sheath is made of a hard material such as hard plastic, silicone, metal or overlay. In this latter embodiment, the outer sheath is designed with a circumference large enough to accommodate the largest dilator and does not itself expand. Instead, it serves as a guide through which the sequential dilators pass to facilitate cervical dilation.

In one particular embodiment, the cervical navigator system comprises two components which are removably attached to each other: a cervical dilating component and a posterior vaginal wall retractor component. These components are designed to connect along grooved tracks that run along both components, with one component containing the “male” connectors and the other containing the “female” receiving components. These connection points are especially important where torque due to pressure on vaginal tissue requires secure attachment to prevent unintended separation during the procedure.

In another embodiment, the posterior vaginal wall retractor component may be manufactured and used separately from the cervical dilator component. This vaginal component encompasses a handle that protrudes from the patient's vagina and is designed to sit comfortably on the patient's buttocks while providing an ergonomic grip for the provider to maintain manipulation and traction. The vaginal wall retractor allows the provider to maintain access to the vagina and cervix without multiple insertions and removals of a vaginal speculum, thereby improving both the patient and provider experiences during gynecological procedures.

In some embodiments the tissue expanding device includes a balloon inflation component that secures all or a portion of the device within the patient by providing an anchoring mechanism at the patient's internal cervical ostia. This balloon is a hollow, flat cylinder that prevents egress of the uterine distending medium, while allowing instruments to pass through its center. By virtue of securing the device to the cervix, it allows for traction of the cervix during manipulation of the vaginal component of the device or vaginal attachment of the device without requiring additional instruments.

In one embodiment, the device comprises a series of sequential cervical dilators that cascade in increasing sizes one over the other. This allows for one dilation of the cervix to the appropriate diameter while needing to identify and traverse the course of the cervix only one time. The sequential dilators may be advanced over an initial small dilator that establishes the pathway through the cervix, with each subsequent dilator having a progressively larger diameter to gradually and safely dilate the cervix to the desired size.

In another embodiment, the cervical navigator device is a medical device including a posterior vaginal wall retractor component that may be attached to the cervical dilator component or manufactured and used separately. This vaginal component encompasses a handle that protrudes from the patient's vagina and is designed to sit comfortably on the patient buttocks and provide an ergonomic handle for the provider to provide manipulation and traction.

In another embodiment, the cervical navigator or medical device further includes a balloon inflation component that allows all or a portion of the device to be secured within the patient by providing an anchoring mechanism to the patient's internal cervical ostia. This balloon is a hollow, flat cylinder that prevents egress of the uterine distending medium, while allowing instruments to pass through its center. In addition, by virtue of securing the device to the cervix, it allows for traction of the cervix during manipulation of the vaginal component of the device or vaginal attachment of the device.

In an alternative embodiment, the cervical navigator system of the present disclosure includes an outer sheath of the component that is anchored within the cervix may be expandable to facilitate passage of sequentially enlarging cervical dilators. (FIGS. 1-3) and a second embodiment in which the outer sheath is a hard material (hard plastic, silicone, metal or overlay) and contains the sequential cervical dilators and is detachably attached to a second component, a vaginal retractor component that is comprised of a more pliable material (soft plastic, silicone, rubber, or overlay).

In yet another embodiment, the cervical navigator and medical device includes a vaginal wall retractor component that passes out of the vaginal and comfortably sits along the patient's buttocks with an area for the provider to grip. This vaginal wall retractor allows the provider to maintain access to the vagina and or cervix without multiple insertions and removals of the vaginal speculum thereby improving the patient and the provider experiences.

In one embodiment, the system of the present disclosure also includes a vaginal retractor component that may contain a mechanism by which to attach the vaginal wall retractor to the patient. In some embodiments, this is achieved by a balloon which is passed through the dilated cervix and then activated to engage a donut shape that allows for instruments to pass through the balloon during engagement. This balloon can be incorporated within the vaginal retractor, inserted within the vaginal retractor or manufactured and distributed separately. While in other embodiments, the medical device and system further includes a cervical balloon that has an open center by which it allows instruments to pass through it while still securely being held within the cervix. Traction on the device will allow for manipulation of the cervix without the use of additional instruments, and balloon inflation will allow for uterine distention with prevention of efflux of uterine distending medium.

In yet another embodiment, the system also includes a series of sequential cervical dilators that cascade in increasing sizes one over the other. This allows for one dilation of the cervix to the appropriate diameter while needing to identify and travers the course of the cervix only one time. While in other embodiments, the system includes two components which are removably attached along grooved tracks which run along both components with one component containing the “male” and one component containing the “female” type of receiving components. These grooves serve as attachment points where torque due to pressure on vaginal tissue requires attachment.

In one embodiment, the cervical navigator system includes an expandable tube which is passed into the cervix and secured with a balloon inflation device and through which the cervical dilators, and then the hysteroscope, are subsequently passed. In one other embodiment, the system also includes a hard plastic, rubber or silicone semi-circle or crescent which itself does not pass through the cervix but is secured to the cervix with a balloon inflation device. This device retracts the posterior vaginal wall allowing for improved cervical exposure during a variety of procedures. It may also provide protection of the posterior vaginal wall during procedures such as vaginal uterine morcellation. It may also contain a handle component which exits the patient's vagina and sits along the patient's buttocks to facilitate the retraction and manipulation.

The present disclosure is a cervical navigator system for gynecological procedures comprising an anchoring portion, an optional dilation mechanism, and a vaginal retraction component. The system includes a balloon inflation unit that anchors at the internal cervical ostia, featuring a donut-shaped design with an open center allowing instrument passage while preventing distending medium efflux. Sequential dilators cascade in increasing sizes over an initial pathway, eliminating the need for repeated cervical navigation. The posterior vaginal wall retractor provides continuous visualization and cervical access without repetitive speculum insertions. Components can be used together or separately, with multiple embodiments including an expandable outer sheath or rigid construction. The device facilitates secure cervical traction without additional instruments and reduces perforation risks by establishing a single, protected pathway. This multi-component system improves patient comfort and procedural safety while providing superior ergonomics for clinicians performing cervical dilation and intrauterine procedures.

The foregoing descriptions of specific implementations have been presented for purposes of illustration and description. They are not intended to be exhaustive or to limit the invention to the precise forms disclosed, and modifications and variations are possible in view of the above teaching. The exemplary implementations were chosen and described to best explain the principles of the invention and its practical application, to thereby enable others skilled in the art to best utilize the invention and its implementations with modifications as suited to the use contemplated.

It is therefore submitted that the invention has been shown and described in the most practical and exemplary implementations. It should be recognized that departures may be made which fall within the scope of the invention. With respect to the description provided herein, it is submitted that the optimal features of the invention include variations in size, materials, shape, form, function, manner of operation, assembly, and use. All structures, functions, and relationships equivalent or essentially equivalent to those disclosed are intended to be encompassed by the invention.

1. A cervical navigator, comprising:
an elongated body having a rigid structure that at least partially deforms in response to contact with living tissue; and
an opening disposed through a center portion of the elongated body; and
at least one dilator removably disposed through the opening that increases a size of the elongated body in response to insertion of the at least one dilator into the opening and at least partial movement through the opening.

2. The cervical navigator of claim 1, wherein the at least one dilator comprises:
a dilator body having a first predetermined diameter; and
a lever disposed at an end of the dilator body to facilitate gripping thereof.

3. The cervical navigator of claim 2, wherein the dilator body moves in response to movement of the lever.

4. The cervical navigator of claim 3, wherein the first predetermined diameter of the at least one dilator is greater than a second predetermined diameter of at least one second dilator.

5. The cervical navigator of claim 1, wherein the at least one dilator is nested within at least one other dilator.

6. The cervical navigator of claim 1, further comprising:
at least one second dilator movably connected to the at least one dilator.

7. The cervical navigator of claim 6, wherein the at least one dilator and the at least one second dilator move telescopically with respect to the elongated body and each other.

8. The cervical navigator of claim 1, wherein the size of the elongated body remains increased after extraction of the at least one dilator from the opening.

9. A method, comprising:
inserting a cervical navigator into a body cavity; and
increasing a size of the cervical navigator in response to insertion of at least one dilator into the cervical navigator and at least partial movement through the cervical navigator, such that the cervical navigator retracts back to its original shape after the dilators are removed and retains capacity to increase in size in response to insertion of instruments therein.

10. The method of claim 9, wherein increasing the size of the outer sheath is based on a first predetermined diameter of the at least one dilator.

11. The method of claim of claim 10, wherein increasing the size of the outer sheath comprises receiving at least one second dilator within a dilator-receiving recess of the at least one dilator.

12. The method of claim 11, wherein increasing a size of the outer sheath comprises simultaneously inserting the at least one second dilator into the outer sheath with the at least one dilator.

13. The method of 9, further comprising:
connecting at least one second dilator to the at least one dilator, such that the at least one second dilator is movable.

14. The method of claim 13, wherein moving the at least one second dilator comprises moving the at least one second dilator and the at least one dilator telescopically with respect to the outer sheath and each other.

15. A medical system, comprising:
a cervical navigator to be inserted at least partially within a body cavity;
a plurality of dilators movably connected within the cervical navigator and extracted from the cervical navigator to dilate the body cavity in response to insertion of at least one of the plurality of dilators into the body cavity;
a retractor removably connected to at least a portion of the cervical navigator to facilitate movement of the cervical navigator; and
an inflation unit removably disposed on at least a portion of the retractor that prevents extraction of the retractor from the body cavity in response to inflation and facilitates insertion of additional instruments.

16. The medical system of claim 15, wherein the retractor is linear along a first portion and, at a second portion, arcuate away from an end of the first portion with respect to a direction.

17. The medical system of claim 15, wherein the cervical navigator comprises a plurality of connector anchors to removably connect to the retractor.

18. The medical system of claim 15, further comprising:
a lever unit disposed on at least a portion of the cervical navigator and connected to each of the plurality of dilators, each of the plurality of dilators moves in response to moving each of the plurality of dilators from a first position to a second position.

19. The medical system of claim 18, wherein the lever unit comprises:
a lever track connected to each of the plurality of dilators; and
a plurality of grooves disposed on at least a portion of the lever track to move each of the plurality of dilators in at least one position on the lever track.

20. The medical system of claim 15, further comprising:
a light disposed on at least a portion of the retractor to illuminate an area around the retractor in response to being turned on.
